(19) United States
(12) Patent Application Publication
(10) Pub. No.: US 2025/0090460 A1
(43) Pub. Date: Mar. 20, 2025

(54) Neurosome/Astrosome Compositions and Methods of Use

(71) Applicant: The Methodist Hospital, Houston, TX (US)

(72) Inventors: Assaf Yosef ZINGER, Houston, TX (US); Caroline Elzabeth Cvetkovic, Houston, TX (US); Robert Conrad Krencik, Pearland, TX (US); Ennio Tasciotti, Houston, TX (US)

(21) Appl. No.: 17/662,657

(22) Filed: May 9, 2022

Related U.S. Application Data
(63) Continuation of application No. PCT/US2020/000043, filed on Nov. 9, 2020.

(51) Int. Cl. A61K 9/1271; A61K 9/51; A61K 31/7105; A61K 31/711; A61K 35/30; A61K 45/06; C12N 15/113

(52) U.S. Cl. CPC A61K 9/1271; A61K 9/5123; A61K 9/5176; A61K 31/7105; A61K 31/711; A61K 35/30; A61K 45/06; C12N 15/113; C12N 2310/14

(57) ABSTRACT

Disclosed are methods for the production and testing of formulated biomimetic nanoparticles. These particles can be utilized towards promoting neural health and connectivity, for example, after injury and in diseased states.

### CROSS-REFERENCE TO RELATED APPLICATIONS

The present application claims priority to PCT Intl. Pat. Appl. No. PCT/US2020/000043; filed Nov. 9, 2020 (pending; Atty. Dkt. No. 37182.248WO01), which claims priority to U.S. Provisional Patent Application No. 62/933,363, filed Nov. 8, 2019 (expired; Atty. Dkt. No.: 37182.248PV01), the contents of each of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND

### Field of the Invention

The present invention relates generally to the fields of molecular biology and pharmacology. In particular, methods are disclosed for the production and testing of formulated biomimetic nanovesicles, termed “neurosomes” or “astrosomes,” which can be utilized in the targeted delivery of therapeutic cargoes to neurological diseases, such as Rett syndrome.

### Description of Related Art

Cellular transplantation has long been proposed as a promising therapeutic avenue for repair or replacement of the nervous system. However, safety and efficacy issues—including uncontrolled proliferation and differentiation, immune response, and off-target effects—make cellular engraftment in the human brain currently unfeasible. The addition of natural or synthetic biomaterials is a promising combinatorial strategy to overcome these challenges.

### DEFICIENCIES IN THE PRIOR ART

The current state of the art does not describe the incorporation of human neuron or astrocyte proteins into nanoparticles, and thus provides little to no guidance for the use of such formulations in therapeutic and/or prophylactic regimens designed to treat neuronal disease, injury, or dysfunction.

### BRIEF SUMMARY OF THE INVENTION

The present disclosure overcomes these and other unmet needs in the art by providing a novel, biomimetic, nano-platform, composed of pluralities of lipid-based nanoparticles that have been functionalized with one or more mammalian neuronal-derived proteins. These functionalized nanoparticles, variously termed “neurosomes,” and “astrosomes” herein, have been shown to be useful in the preparation of medicaments for therapy of nervous system disorders, and the like, and provide new approaches for neuroregeneration and treatment of neural diseases and the like.

### BRIEF DESCRIPTION OF THE DRAWINGS

The following drawings form part of the present specification and are included to demonstrate certain aspects of the disclosure. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, and FIG. 1F show neural biomimetic NV synthesis and physiochemical characterization. (FIG. 1A) Human pluripotent stem cells (hPSCs) were either cultured in vitro or differentiated for 7 days to iNeurons for membrane protein extraction. (FIG. 1B) A stable membrane-bound green fluorescent protein (memGFP) transgene was incorporated. Scale: hPSCs, 500 μm; iNeurons, 100 μm. (FIG. 1C) A microfluidic approach was utilized for the synthesis of neural biomimetic NVs with cell-specific membrane proteins. Two different lipid formulations (A or B) were used to fabricate the NVs. Thus, three NV groups were fabricated using each lipid formulation: liposomes (Lipo-, L), plurisomes (Pluri-, P) and neurosomes (Neuro-, N). (FIG. 1D) Cryo-TEM images demonstrated that all NV formulations had similar spherical lipid bilayer morphologies. Scale: 50 nm. (FIG. 1E) Immunoblotting revealed transfer of mem-GFP in NVs originating from hPSCs and iNeurons, and transfer of neuronal membrane protein marker NCAM1 in NVs originating from iNeurons only. (FIG. 1F) Physiochemical properties including NV size, PDI and zeta potential were assessed. Though neither NV size nor PDI were significantly altered between NVs of different lipid formulations, NVs from lipid formulation B displayed a less negative zeta potential. Results are shown as mean±SEM (n=3-7; see FIG. 5B). One-way ANOVA followed by Tukey's multiple comparison test was used to determine statistical probabilities between NVs of different protein sources within the same formulation (p>0.05 for all results shown in this figure);

FIG. 2A, FIG. 2B, and FIG. 2C show cellular association of biomimetic NV in vitro. (FIG. 2A) memGFP-iNeurons were incubated with rhodamine-labeled NVs for 24 hours to qualitatively confirm targeting efficiency and uptake of liposomes, plurisomes, and neurosomes of both formulations A (top) and B (bottom) with confocal microscopy. Scale: 10 nm. (FIG. 2B) Neither lipid formulation of neurosomes or liposomes resulted in significant cytotoxicity at 100 M up to 48 hrs of treatment time compared to untreated cells, as determined by a viability assay (see FIG. 6B). (FIG. 2C) High throughput fluorescence-activated cell sorting (FACS) was utilized to quantitatively assess NV-iNeuron association (n=3-6). Data are presented as fluorescent intensities normalized to L_{A} iNeurons exhibited significantly increased preferential association with N_{A} compared to L_{A} (p=0.014). One-way ANOVA followed by Tukey's multiple comparison test was used to determine statistical probabilities between NVs of different protein sources within the same formulation in FACS experiments(*=p<0.05). Results from formulation A are displayed in the top half of the figure, whereas results from formulation B are displayed in the bottom half of the figure;

FIG. 3A and FIG. 3B show humanized NV association within 3D sphere culture. 24-hour treatment with NVs from lipid formulation A (FIG. 3A) or B (FIG. 3B) at 100 or 500 M did not considerably reduce cellular viability of iNeurons cultured in 3D spheres compared to non-treated controls, as determined by a CellTiter-Glo 3D Assay (n=8-10 spheres per group). Luminescence readings were normalized to cross-sectional areas of iNeuron spheres;

FIG. 4A, FIG. 4B, FIG. 4C, FIG. 4D, and FIG. 4E show neural NV synthesis. (FIG. 4A) Targeting scheme used to generate membrane-bound green fluorescent protein (memGFP)-containing, dox-inducible iNeurons. (FIG. 4B) Membrane protein (MP) as a function of starting cell density (millions of iNeurons), extracted using a ProteoExtract® Native Membrane Protein Extraction kit and quantified using a Pierce Rapid Gold BCA kit. iNeurons provided an average of 15.7±2.3 μg of MP per million cells. (FIG. 4C) Macroscopic Cryo-TEM demonstrated that all NV formulations had similar spherical lipid bilayer morphologies. Scale: 100 nm; see FIG. 1D. (FIG. 4D) Coomassie blue staining after SDS-PAGE separation confirmed transfer of proteins from cell lysate to membrane fraction into NVs. Column labels are shown at right. (FIG. 4E) Immunoblotting of GFP and NCAM1 proteins;

FIG. 5A and FIG. 5B show neural NV characterization. (FIG. 5A) NanoSight quantification of liposome, plurisome, and neurosome concentrations (particles per mL) from formulation A. Bold lines indicate averages; shaded areas represent SEM. (FIG. 5B) Detailed results (mean values±SEM) of physiochemical characterization of NVs from Formulations A and B, including NV size, polydispersity index (PDI), and zeta potential, measured using a Zetasizer Nano ZS system (see FIG. 1F);

FIG. 6A, FIG. 6B, and FIG. 6C show cellular association of NVs. (FIG. 6A) Two representative control images from qualitative confocal microscopy of untreated mem-GFP cells (see FIG. 2A). Scale: 10 μm. (FIG. 6B) Extended cellular viability results for 24- to 72-hr incubation periods at concentrations ranging from 100 μM to 1 mM (normalized to untreated cells). An increase in NV concentration surpassing 500 μM in formulation B led to significantly decreased viability over time. A 2-way ANOVA was performed, followed by Dunnett's multiple comparison test (comparing increasing NV concentrations to untreated cells within NV formulations A or B) or Tukey's multiple comparison test (comparing protein sources, within NV formulations A or B, across increasing concentrations). (FIG. 6C) Fluorescent intensity (a.u.) of NVs from formulations A and B. Detailed results (mean values±SEM) of normalized fluorescent intensity from quantitative high throughput fluorescence-activated cell sorting (FACS) are shown in FIG. 2C. Results are shown as mean±SEM. One-way ANOVA followed by Tukey's multiple comparison test was used to determine statistical probabilities from FACS experiments. *=p≤0.05, **=p≤0.01;

FIG. 7 shows that Dexamethasone (Dex) can be encapsulated inside the disclosed nanovesicles (liposomes and neurosomes) using the microfluidic approach described herein;

FIG. 8 shows that by increasing the initial stock concentration of Dex, the final encapsulation can be increased by as much as ˜3 fold;

FIG. 9 shows a schematic of a biomimetic nano-platform, composed of lipid-based nanoparticles functionalized with human neuronal-derived proteins, in accordance with one aspect of the present disclosure to target specific cell types within the human nervous system; and

FIG. 10 shows a schematic of the methods for producing iNeurons from hPSCs, cell membrane protein extraction, benchtop microfluidic assay to incorporate proteins into the lipid-based nanoparticles and exemplary materials characterization in accordance with one aspect of the present disclosure.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

### Astrocytes

Astrocytes are highly abundant non-neuronal glial cell types in the nervous system that maintain functional homeostasis of neural networks. After injury, disease, and infection, glial cells react and can contribute inflammatory signaling molecules to the surrounding micro-environment, in turn, negatively impacting neural function. How can therapeutics, such as anti-inflammatories, be selectively delivered to these inflammatory astrocytes in order to reduce off-target drug effects on other cells? At present, there are no effective clinical approaches for this purpose.

The present invention seeks to overcome this lack of technology by providing nanovesicles that demonstrate an enhanced inflammatory astrocyte-restricted delivery of a therapeutic cargo to a selected neural cell or tissue. By integrating membrane proteins from human pluripotent stem cell-derived inflammatory astrocytes into the surface of lipid-based nanovesicles, cell-specific targeting of the contents of the resulting nanovesicles could be preferentially directed to neural cells of interest. Nanovesicles coated with adhesion molecules derived from inflammatory astrocytes [a.k.a., “AstroVesicles” (AVs)] will bind to protein-interacting partners, specifically at the surface of inflammatory astrocytes and, thus, increase cellular uptake of therapeutic cargo.

By developing novel human biomimetic nanotechnology “AstroVesicles” for improved targeted delivery of therapeutics to subtypes of inflamed brain cells, the inventors have tested whether incorporating inflammatory astrocyte-derived proteins into lipid-based nanovesicles enhanced targeting to inflammatory astrocytes within a multicellular human sphere culture platform. The resulting invention yields next-generation tools capable of more effective reduction of neuroinflammation.

### Nanovesicles

Nanovesicles (NVs) are emerging tools for therapeutic cargo delivery. Surface engineering of NVs through incorporation of membrane proteins from specific cell types offers a promising route to enable complex functional attributes. However, this type of biomimetic approach has not yet been explored using human neural cells for utility towards the nervous system. Thus, here the inventors have optimized and validated a method for scalable and reproducible production of NVs incorporated with membrane proteins unbiasedly sourced from human pluripotent stem cell (hPSC)-derived neurons. These studies established that endogenous and transgenic proteins can be transferred from cells to NVs without disruption of NV properties nor human neuron viability, as determined using monolayer cultures and three-dimensional spheres. Further, NVs with neuron-membrane proteins exhibited enhanced neuronal association and uptake. Finally, the temporal bioavailability of humanized NVs was defined within the rodent brain. In summary, these customizable NVs enable a next generation of functionalized therapeutics for neuroregeneration.

### Traumatic Brain Injury

Restoration of neural function after traumatic brain injury (TBI), neurodegeneration, or neuroinflammation is currently hindered by a lack of effective and clinically practicable biotechnologies for precise cell-targeted therapy. Breakthroughs are needed for enhanced and sustained delivery of therapeutic cargo (e.g., genetic material and chemical compounds with nanomaterials while also mimicking properties of the brain. Above all stands the challenge of avoiding the potential side effects that can occur with methods such as viral delivery. Nanotechnologies that are inspired from nature, known as bio-inspired or biomimetic tools, are one approach to gain insight into safe and tractable methodologies). For example, cell-derived exosomes are promising drug delivery systems; however, new approaches are needed as the complexity and variability of biomimetic tools from cellular sources reduces their potential for scalable use in precision medicine.

### Functionalized Nanoparticles

Functionalized nanoparticles (NPs) have emerged as potential well-defined carriers for selective and targeted delivery of cargo to neural cells due to their size scale. For example, NPs have been used for functional delivery of drugs to the rodent brain in multiple pathologies. Various surface modifications, such as coupling targeting peptides or antibodies to NPs or modifying surface charge for selective neuron-specific targeting, have been employed to increase targeting efficacy. Alternatively, exosome-like lipid nanovesicles (NVs) have also been used for delivery to the brain as a means to mimic neural cellular communication. However, standardized protocols for their storage and characterization have not yet been fully established and low yield from biological sample sources reduces the potential of translating this platform to the clinic.

In an effort to benefit from the advantages offered by both native cells and synthetic NPs, the inventors previously pioneered hybrid biomimetic nanoparticles that integrate leukocyte cell membrane proteins into lipid-based nanovesicles to achieve enhanced bioactivity to specific cell types while maintaining functionality. For example, it was demonstrated that the incorporation of leukocyte-derived plasma membrane proteins into spherical bilayered phospholipid NPs enables immune system avoidance and functional communication with inflamed endothelial cells. This biomimetic approach is thought to occur through transfer of cellular adhesion proteins to the surface of NVs and thus promote protein-protein interactions with target cells. A similar approach for targeting neural cell types has potential, as it is known that cellular interactions of neurons are at least partially based on cell-cell binding of adhesion proteins presented at the cell membrane surface. Nonetheless, testing this approach with human neural cells is hindered due to the lack of pure human neural cell sources for reproducible and scalable production. However, recent advances in human pluripotent stem cell (hPSC) differentiation into specific neural cell types may now enable the generation of biomimetic NVs and experimental testing platforms that are both scalable and more appropriate for clinical translation as compared to using rodent-derived neural cells.

Based on this premise, the inventors have developed and defined a new class of biomimetic human neural NVs (a.k.a. “neurosomes”) using a reproducible and scalable protein source from a pure population of hPSC-derived excitatory cortical neurons. Using a microfluidic-based synthesis method, the inventors have bioengineered proteolipids with these membrane proteins into NVs. It was shown that incorporation of neuron-derived membrane proteins does not affect the physicochemical properties of NVs and enhances their uptake into cultured neurons. The inventors further confirmed proof-of-principle therapeutic efficacy with human neural sphere cultures and the rodent nervous system. Finally, candidate adhesion proteins were identified in neurosomes which will pave the way for fully synthetic nanovesicle formulations. These studies advance the current paradigm of NV bioengineering for improved cellular targeting within the nervous system.

### Pharmaceutical Formulations

In certain embodiments, the present invention concerns methods and electrospun collagen-base implantable delivery systems suitable for delivery of therapeutic, diagnostic, or prophylactic agents to one or more cells or tissues of an animal, either alone, or in combination with one or more other modalities of diagnosis, prophylaxis and/or therapy. The formulation of pharmaceutically acceptable excipients and carrier solutions is well known to those of ordinary skill in the art, as is the development of suitable surgical implantation methods for using the particular compositions described herein in a variety of treatment regimens.

Sterile injectable compositions may be prepared for storing the disclosed implantable delivery systems using appropriate solvent(s) alone, or including one or more additional ingredients using conventional methods. Generally, dispersions can be prepared by incorporating the selected sterilized active ingredient(s) into a sterile vehicle that contains the basic dispersion medium and the required other ingredients from those enumerated above. The delivery systems, membranes, and patches disclosed herein may also be formulated in solutions comprising a neutral or salt form to maintain the integrity of the systems, membranes, and patches prior to implantation.

Pharmaceutically acceptable salts include the acid addition salts (formed with the free ammo groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2nd Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2nd Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible nanovesicle or nanoparticle.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

The terms “for example” or “e.g.,” as used herein, are used merely by way of example, without limitation intended, and should not be construed as referring only those items explicitly enumerated in the specification.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

The term “pharmaceutically-acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cell. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Praline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “_{L}” isomeric form. However, residues in the “_{D}” isomeric form may be substituted for any _{L}-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, “porosity” means the ratio of the volume of interstices of a material to a volume of a mass of the material.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 g/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 g/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be an anesthetic, an analgesic, a chemotherapeutic agent, an immunosuppressive agent, a cytokine, a cytotoxic agent, a therapeutic polypeptide or polynucleotide, a proteolytic or nucleolytic compound, a radioactive isotope, a receptor, an enzyme, or a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may also be delivered using one or more of the disclosed scaffolds or matrices. For a more detailed description of anticancer agents and other therapeutic agents, those skilled in the art are referred to any number of instructive manuals including, but not limited to, the Physician's Desk Reference and the work

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to deliver one or more nucleic acid segments using a delivery system disclosed herein, including, for example, in combination with an appropriate detectable marker (i.e., a “label,”). A wide variety of appropriate indicator compounds and compositions are known in the art for labeling polynucleotides and polypeptides, including, without limitation, fluorescent, radioactive, enzymatic or other ligands, such as avidin/biotin, etc., which are capable of being detected in a suitable assay or observed in situ. In certain embodiments, it may be desirable to include one or more fluorescent labels or an enzyme tag such as urease, alkaline phosphatase or peroxidase, instead of radioactive or other environmentally less-desirable reagents as part of the delivery system.

In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

### Biological Functional Equivalents

Modification and changes may be made in the structure of a nucleic acid, or to vectors comprising it, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| | AMINO ACIDS | CODONS | | | | | |
|---|---|---|---|---|---|---|---|
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, specifically incorporated herein in its entirety by express reference thereto). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine(+1.9); alanine(+1.8); glycine (−0.4); threonine (−0.7); serine (−0.8); tryptophan (−0.9); tyrosine (−1.3); praline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101 (specifically incorporated herein in its entirety by express reference thereto), the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); praline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biological equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### EXAMPLES

The Examples attached hereto are included to demonstrate preferred embodiments of the invention. It should be appreciated by those of skill in the art that the techniques disclosed in the accompany examples represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Biomimetic Targeting of Neural Cells with Nanoparticles

Nanoparticles (NPs) have been long investigated for the selective delivery of therapeutics and for the avoidance of off-target effects. However, once administered into the body, NPs face both physical and biological barriers that impede the capability of reaching the diseased tissues at therapeutic levels. Current strategies to modify the surface of NPs with targeting antibodies or hydrophilic polymers have not yielded effective results and have failed to translate to the clinic, leaving the entire field of nanomedicine in dire need of an alternative approach.

In this example, a new class of biomimetic lipid-based NPs have been developed, which are enriched with membrane proteins derived from human neural cells (i.e., neurons, astrocytes, etc.). These nanoparticles have been termed “neurosomes.” Based on published literature on the trafficking of exosomes between neural cell populations and these in vitro and in vivo data, it was successfully demonstrated that neurosomes improve neural targeting and delivery of a relevant therapeutic cargo to selected target cells of interest in mammalian systems.

These neurosomes were prepared by a process that employed the NanoAssemblr™ Benchtop (Precision Nanomedicine) by applying extensive expertise in synthesizing biomimetic NPs using microfluidics. In this process, membrane proteins from human pluripotent stem cells (hPSCs), hPSC-derived astrocytes, and hPSC-neurons have been extracted, and integrated with specific lipids using such microfluidic approaches. In addition, the characteristics of these neurosomes are preserved by maintaining reproducibility (batch-to-batch and consistent protein presentation) and stability to retain surface proteins. The following approaches were most successful in achieving the populations of neurosomes with highly-desirable properties:

Neurosome lipidformulation: Multiple combinations of lipid formulations were tested. The described lipids were chosen based on their ability to load mRNA and to support the loaded nanoparticle structure that preferably included one or more of these lipids as the NPs backbone:
D-Lin-MC3-DMA
1,2-dipalmitoyl-sn-glycero-3-phosphocholine (DPPC)
1,2-distearoyl-sn-glycero-3-phosphocholine (DSPC)
1,2-dioleoyl-sn-glycero-3-phosphocholine (DOPC)
1,2-dioleoyl-sn-glycero-3-phosphoethanolamine (DOPE)
1,2-distearoyl-sn-glycero-3-phosphoethanolamine-N-[amino(polyethylene glycol)-(ammonium salt) (PEG-2000) and
1,2-dipalmitoyl-3-dimethylammonium-propane (16:0 DAP) Cholesterol

Reproducibility: The reproducibility of the physical characteristics of NPs (liposomes and neurosomes) have been assessed by monitoring variations in size polydispersity index (PDI), zeta potential and concentration using a Zetasizer Nano ZS particle D.

The stability of NPs at 4° C. was assessed, and it was found it inadequate i.e. ±40 nm size, ±5 mV zeta potential and ±0.2 PDI.

The NPs association towards neurons and astrocytes were increased by adding neuron and astrocyte-derived membrane proteins.

Genetic cargoes have been protected from cellular degradation by encapsulating them into NPs.

A variety of genetic cargoes have been shown to be amenable to encapsulation within various populations of these NPs.

Additionally, at least one of the key components responsible for NPs association—the NCAM1 integrin—has been identified and characterized. Likewise, the NPs toxic effect of neural organoids can be assayed using the multielectrode array techniques described herein.

### Methods and Results

Neurosome Generation. A pure population of iNeurons was derived from hPSCs following established protocols (Evangelopoulos et al., Nanomaterials, 2018); briefly, on day 7, membrane proteins were extracted and purified using a commercial kit. Lipid-based nanoparticles incorporating iNeuron-derived proteins (e.g., Neurosomes) were generated using a microfluidic assay that utilizes a benchtop NanoAssembler™ system (Krencik et al., Stem Cell Rep., 2017). Particles were also generated from hPSC-derived membrane proteins (Plurisomes) and without incorporated protein (Liposomes) as controls.

Material Characterization. Physical characteristics and reproducibility were assessed with NanoSight NS300, Dynamic Light Scattering Zetasizer Nano ZS, and cryo-transmission electron microscopy. Mass spectrometry and Western blot confirmed retention of well-known cell source-restricted proteins between Neurosomes and Plurisomes. No significant differences in nanoparticle size, polydispersity index, or charge (zeta potential) were observed between groups.

In vitro Studies. Monolayers of iNeurons were cultured for 7 days. After 24 hrs of treatment, cells were either evaluated with a MTS metabolic assay to assess potential cellular toxicity or dissociated for flow cytometry and microscopy. iNeurons did not display significant cytotoxicity in the presence of neurosomes and liposomes. Flow cytometry and confocal microscopic imaging indicated that iNeurons exhibited higher association with neurosomes compared to plurisomes or liposomes (Hoffman et al., Methods Mol. Biol., 2018).

These studies demonstrated the capacity of systematically recapitulating the biological complexity of neuronal cell membranes onto nanoparticle surfaces, which allows for full characterization of their biological and physical properties. Moreover, this top-down nanotechnology is high yield, cost-effective, and scalable. A variety of optimized formulations from these studies are also readily transferrable into existing preclinical animal model experiments. The disclosed drug delivery platform represents a feasible alternative to cellular transplantation, which can be a useful nanotechnology in the regenerative medicine community for targeting of cellular populations and delivery of small molecules, fluorescent tracers, growth factors, imaging contrast agents, or genetic cargo to the injured or diseased nervous system.

### Example 2—Nanoparticle Targeting of Neurons to Treat Rett Syndrome

Rett syndrome is a devastating disease that takes effect during neurological development and predominantly afflicts pre-adolescent females. As the symptoms manifest, neural function regresses, and patients display abnormalities, including loss of motor function, extreme anxiety, hyperventilation, and other issues that severely reduce quality of life. Various mutations in the methyl CpG binding protein 2 (MeCP2) gene lead to loss of functional protein and altered changes in gene expression, which is responsible for the initiation and progression of the disease. MECP2 is found on the X chromosome. Because only one copy of the X chromosome is active in any given female cell, only a fraction of cells is affected by the MECP2 mutation, and the severity and progression of Rett syndrome is correlated to the percentage of cells expressing the mutant MECP2 gene.

Several approaches have been designed to treat and/or ameliorate one or more symptoms of Rett syndrome by attacking the lack of the MeCP2 protein, which is at the root of the disorder. Gene therapy approaches aim at introducing functional copies of the MECP2 gene to restore normal levels of MECP2 but are limited due to nonspecific delivery approaches and clinical safety issues concerning the use of viral vectors. Rett Syndrome Research Trust is currently funding work with AAV2 and AAV9 vectors in a mouse model of Rett Syndrome. However, while AAV therapies have proven to be very effective in animal models, there are no clinical trials in which efficacy has occurred. Protein replacement therapy aims at directly providing the MeCP2 protein thus avoiding the issues linked to viral vectors, number of copies of gene transferred, insertional mutagenesis, etc. However, this approach will require multiple protein administration and is also lacking the use of specific targeting.

Ultimately though, both approaches are still in early development, and share the same primary challenge; that is, delivery of either MECP2 genes or protein requires overcoming systematic barriers to introducing a foreign biological material into cells. Direct injection of genes or proteins is inefficient, as degradation may occur prior to the uptake into target cells. Hence, novel methods that can protect either the gene or the protein from enzyme degradation, as well as deliver the encapsulated cargo directly to neurons, are urgently needed for the effective treatment of Rett syndrome.

In this example, the development and testing of a completely new nano-approach to targeted delivery of a therapeutic cargo is detailed. The nanoparticles are based on the use of membrane proteins extracted from neuronal cells to achieve selective targeting and preferential uptake. These studies provide valuable information relevant for the delivery of a variety of therapies directed to treating the symptoms of brain dysfunction; ultimately, such therapeutics may be central to developing a cure for this devastating syndrome.

As noted above, nanoparticles (NPs) have been long investigated for the selective delivery of therapeutics and for the avoidance of off-target effects. However, once administered into the body, NPs face both physical and biological barriers that impede the capability of reaching the diseased tissues at therapeutic levels. Current strategies to modify the surface of NPs with targeting antibodies or hydrophilic polymers have not yielded effective results and have failed to translate to the clinic, leaving the entire field of nanomedicine in dire need of an alternative approach. If proven effective, the use of this platform may benefit other researchers engaged in the discovery of treatments for diseases such as Rett syndrome, whether the treatment involve the delivery of a drug, a genetic construct (e.g., gene) or a polypeptide or protein of interest.

Due to the lack of viable therapeutic targets for the treatment of Rett syndrome, it is essential to gain insights into potential new treatment options for the treatment of this disease. This proposal will build upon the tremendous expertise of the team of investigators and elegantly synergize a technology platform that allows for the targeted delivery of mRNA agents to specific cells. The assessment of the therapeutic efficacy and safety of molecularly targeted therapies will provide valuable insights into the clinical applications of neurosomes and their ability to deliver mRNA to improve the Rett syndrome patient's quality of life.

It is hypothesized that neuron-based biomimetic NPs will yield an efficient and specific delivery of wild-type MECP2 mRNA to neurons without changing the germ cells. If successful, this new therapeutic delivery tool can be subsequently tested in pre-clinical animal trials to restore MeCP2 expression in neurons. The inventors believe that neuron-based biomimetic NPs will (1) improve neuronal targeting, (2) enhance the accumulation of neurosomes in neurons compared to conventional liposomal-based platforms, and (3) increase the expression of normal MeCP2 protein in neurons. Taken together, these data indicate that the innovative compositions and methods disclosed herein may be translated towards clinical trial testing to rescue impairments in patients with Rett syndrome.

### Example 3—Specialized Leukosomes for Targeting of Neurons

Inspired by the leukocytes' evasion of immune cell sequestration, tropism towards inflamed endothelium, and infiltration of the cancer mass, a new generation of biomimetic NP has been previously developed by some of the present inventors. These particles were termed “leukosomes.” Leukosomes are liposome-like NPs formulated with purified membrane proteins extracted from circulating leukocytes. The method of synthesizing leukosomes conserves the versatility and physicochemical properties of liposomes, including the ability to load compounds with various solubility and show multiple functionalities due to the simultaneous surface modification with multiple leukocyte membrane proteins. It has been shown that leukosomes escape immune cell uptake, increase circulation time, target activated vessels and inflamed tissues, and possess intrinsic anti-inflammatory activity.

In this example, the extensive experience gained during the development of specialized leukosomes for the targeting of localized injury, sepsis, and atherosclerotic plaques has be exploited to develop a new class of biomimetic lipid-based NPs enriched with membrane proteins derived from neurons. As noted above, these particles have been termed “neurosomes” (NPs).

Based on published literature on the trafficking of exosomes between neuronal cell populations and preliminary data in vitro and in vivo reported below, it was hypothesized that neurosomes would demonstrate improved neural targeting and be capable of delivering therapeutically-relevant cargo to neuronal cells for the treatment of various neural disorders. Using this innovative strategy, the incorporation of neuron-derived membrane proteins promote specific homotypic/homophylic adhesion to the neuronal surface and thus increase targeted delivery and selective uptake. Such neurosomes display unique biological properties due to the presence of adhesion membrane proteins derived from neurons, and retain the pharmacological versatility of liposomes, thus enabling the delivery of drugs with various chemical properties (e.g., hydrophilic, and lipophilic) and genetic cargoes (e.g., DNAs, mRNA, miRNA, siRNA). Such neurosomes can be rapidly translated to clinical applications as they can be manufactured using similar, standardized manufacturing protocols (scalability, good manufacturing practices) previously established for liposomes. The use of biomimetic NPs provides a new and innovative approach to the targeted delivery of therapeutic cargoes to neurons and selected neural tissues.

Biomimetic strategies represent a paradigm shift in the design of NPs, enabling next generation platforms capable of effectively interfacing and interacting with complex biological systems. Neurosomes bridge the gap between synthetic (liposomes, polymeric particles, etc.) and biological NPs (exosomes, ectosomes, etc.). Neurosomes substantially advance the current paradigm for NP targeting and shielding, which rely on synthetic routes (e.g., functionalization with antibodies, peptides or hydrophilic polymers). Currently most methods for the production of NPs are based on the assembling of individual building blocks (i.e., a “bottom-up” approach), into NPs that are then surface-functionalized using ligands and other molecules. These approaches require multiple synthetic routes and purification protocols that complicate the fabrication increase costs, lowers yields and result in a very complex (when not impossible) scaling up. Even more so, current chemical conjugation methods remain unable to reproduce on the surface of NPs the biological complexity of the cellular membrane. The methods presented here demonstrate a radically different approach to the assembly of complex multifunctional NPs, and provide new modalities that pioneer the use of high yield, cost-effective, scalable, “top-down” synthesis methodologies to recapitulate the biological complexity of cell membranes.

Neurosome lipidformulation: Multiple combinations of lipid formulations will be tested. The described lipids were chosen based on their ability to load mRNA and to support the loaded nanoparticle structure including these lipids as the NPs backbone.

Reproducibility: The reproducibility of the physical characteristics of NPs (liposomes and neurosomes) may be assessed by monitoring variations in size, polydispersity index (PDI), zeta potential, and concentration using a Zetasizer Nano ZS particle analyzer, NanoSight NS-300 nanoparticle characterization system, and cryo-transmission electron microscopy (TEM).

Protein consistency: This parameter is typically analyzed using micro-BCA assay, denaturing polyacrylamide gel electrophoresis (SDS-page), mass spectrometry, Western blot (WB) and/or cryo-TEM in combination with specific fluorescent and metal-labeled antibodies. Briefly, NPs surface-specific receptors will be quantified using WB or cryo-TEM identifying receptors with fluorescent and metal-labeled antibodies, respectively. Human neuron cell lines that stably express a membrane-targeted green fluorescent protein (mGFP) may be used as the base for developing neurosomes.

Stability: Stability is assayed by evaluating the changes in size, zeta potential, NP concentration, and the expression of specific biomarkers using WB for up to 21 days. One can assess the stability at two different temperatures: 4° C. (storage temperature) and 37° C. (physiological temperature).

The encapsulation efficiency of scrambled mRNA: As an initial proof-of-concept for mRNA loading work flow, GFP-encoding mRNA is loaded into neurosomes. The encapsulation efficiency will be determined using Quant-iT™ RiboGreen® RNA Reagent and Kit (Invitrogen) assay. The fluorescent protein expression will be evaluated using IncuCyte® Live Cell Analysis Systems. Different cholesterol amounts can be tested in order to determine the optimal mRNA release rates. The neurosomes' cholesterol content in the membrane can be varied (e.g., 0, 15, or 30 mol %) to test the effect on prolonging the mRNA release rate at 37° C. Determination of an optimal formulation of neurosomes that yield a reproducible size, zeta potential, PDI, mRNA encapsulation efficiency and membrane protein consistency may be evaluated by specific neural membrane proteins such as ICAM1, L1CAM, and the like. Where stability of NPs at 4° C. is inadequate i.e., ±40 nm size, ±5 mV zeta potential and ±0.2 PDI, a number of modifications may be made to achieve compositions with therapeutic properties. For example, (1) adjusting the formulation to incorporate longer lipid chains that will stabilize the formulation, (2) increasing the molar concentration of saturated lipid chains; and/or (3) increasing the concentration of cholesterol. Similarly, in applications where the encapsulation of a selected mRNA is too low, lower cationic lipid:mRNA ratios can be substituted to produce a more useful therapeutic.

The most stable and reproducible formulation for neurosomes may be selected to investigate the viability and targeting of particular types of neurons in vitro. The viability of neurons after treatment with NPs (neurosomes and liposomes) can readily be quantified with an MTS cell viability assay comparing different NP concentrations (e.g., 10, 25, 50, 100, 250 and 500 M) at three different time intervals (e.g., 24, 48 and 72 hr). The percentage of viable cells will be calculated in relation to the untreated wells. The highest concentration where cells maintained >90% viability will be used as the working, therapeutic concentration of neurosomes.

Neuronal targeting of neurosomes may be determined using traditional human neuronal monolayer culture (i.e., 2D) and organoid co-culture system composed of both neurons and astrocytes (i.e., 3D); in such studies, neurosomes may be compared to control synthetic NPs (liposomes). The association and internalization of neurosomes by neurons can be evaluated in 2D (neurons only) by confocal microscopy and flow cytometer using rhodamine (1,2-dioleoyl-sn-glycero-3-phosphoethanolamine-N-(lissamine rhodamine B sulfonyl)-labeled NPs. These and similar studies can further the understanding of the internalization process of NPs and lead to second-generation derivatives having improved properties. Rhodamine NPs will be incubated with neurons at various concentrations (1-500 M). Samples after 1, 4, 12, and 24 hrs of incubation will be assessed for NP uptake and internalization using confocal microscopy.

The 3D co-culture system is an improved model system of the brain environment, more physiologically relevant and predictive than 2D cultures. Moreover, this 3D system will better model the complex conditions in a living organism. Thus, the specificity of neurosomes targeting neurons over astrocytes will be examined in 3D human organoids. Rhodamine-labeled NPs may be incubated with the 3D system model using the optimal concentration and incubation time discovered in the 2D experiments as initial conditions. For imaging, confocal microscopy may be used for 3D acquisition and analysis.

It was expected that increased neuronal targeting along with minimal toxicity using the neurosomes formulation on both the 2D and 3D cells model when compared to the liposome-treated cells. This higher targeting will lead to higher GFP expression in the neurons.

In the case of inadequate targeting of neurons while comparing it to liposomes targeting, one may investigate if incorporating higher concentrations of the extracted membrane proteins during the synthesis of neurosomes. It is hypothesized that higher concentrations of extracted membrane proteins will result in more efficient targeting in particular applications of this new technology.

In the studies shown herein, data is presented as means and standard error of the mean (SEM) of a minimum of three biological replicates consisting of three technical replicates each. The Kolmogorov-Smirnov test was used to test for normal distribution. To analyze the differences between groups, different analysis tests may be employed as necessary. Data with normal distribution can be analyzed using one-way ANOVA with Bonferroni's multiple comparison test, while data with non-normal distribution can be analyzed and reported using the Kruskal-Wallis test. Such analyses may be performed using the statistical software package “Graph Pad Prism,” with a statistical significance defined as p<0.05.

### Example 4—“Astroparticles” for Treating Stroke or Brain Injury

Astrocytes are a highly abundant non-neuronal cell type in the nervous system that contribute to the formation and strengthening of synaptic networks. Although it is clear that astrocytes produce extracellular synaptogenic proteins during neurodevelopment, there are currently no therapeutic approaches to provide these proteins in the mature brain to promote neural connectivity after traumatic injury and stroke. Cellular transplantation of astrocytes is one promising strategy, yet there are numerous safety concerns that make this currently unfeasible. Here, in this example, the inventors overcome this issue by generating an innovative method to deliver human astrocyte-derived synaptogenic proteins for regenerating neural networks without causing cellular toxicity. For the first time, a bio-inspired tool named ‘Astroparticles’ have been formulated by combining human pluripotent stem cell-derived astrocyte membrane proteins into the surface of lipid-based nanovesicles. Astroparticles can be compared to traditional nanovesicles comprising one or more proteins from other cellular sources as a control. The reproducibility and stability of their physical characteristics can also be readily assayed, and their effect upon human neural culture viability can be determined. Likewise, the capability of Astroparticles to induce synaptic formation and strength can be demonstrated using a three-dimensional coculture system composed of human astrocytes and optogenetic neurons. Astroparticles represent an effective, non-destructive, therapeutic that can be readily adapted for use in a variety of therapies for use in neuroscience as a means to promote synaptic connectivity. The incorporation of proteins into nanovesicles is expected to reduce diffusion, improve stability and permit systemic injection within the body as compared to solely using proteins.

Another important aspect of the invention is the development of novel human biomimetic nanotechnology (e.g., “Astroparticles”) for promoting synaptic connectivity after injury. Specifically, human astrocyte-derived proteins when formulated into the lipid-based nanovesicles represent an effective tool for improving the synaptic connectivity with an innovative human neural network testing platform.

### Example 5—Astroparticles as Therapeutics for Neuroregeneration

Astrocytes are highly abundant throughout the nervous system and make significant contributions to neural network development, maturation and homeostasis. However, the ability to translate knowledge of astrocytes into clinical interventions, such as restoration of neural connectivity after brain injury, is hindered by a lack of effective technologies. The inventors have previously devised methods to produce astrocytes from human pluripotent stem cells (hPSCs) and confirmed their ability to xenograft into the brain as a potential cellular therapy. However, cellular brain transplantation into patients is currently unfeasible because of safety concerns. Bio-inspired theranostic nanotechnologies are an alternative means to deliver cell-derived components to the brain via functionalized nanoparticles.

### Formulation of Astroparticles

Following the same strategy used to produce and characterize Neurosomes and Plurisomes as described above, Astroparticles may also be generated by combining day-14 iAstro protein extracts with the same lipid formulations on the NanoAssemblr. As described above, the physiochemical parameters of Astroparticles including size distribution, concentration, zeta potential, and morphology may be measured using the NanoSight NS300, Dynamic Light Scattering Zetasizer Nano ZS and TEM. Lipid consistency is tested with the modified Stewart lipid determination assay and thin layer chromatography (TLC). Stability is tested at two different temperatures (4° C. and 37° C.) and with incubation in 5 buffers (water, phosphate buffered saline (PBS), fetal bovine serum (FBS), 10% sucrose and 50/50 PBS/FBS) for 21 days. The stability of Astroparticles at these conditions is investigated by measuring the analytical parameters described above. Mass spectrometry-based proteomics and Western blot can be used to confirm retention of cell source-restricted protein identifiers.

The impact of Astroparticles on viability of iNeurons, iAstros and Asteroids can be assayed at various concentrations (e.g., 0, 10, 50, 100, 500 M). Cell viability can be evaluated using an MTS assay at 24 hr, 1 week and 3 weeks after treatment at each media replacement. LIVE/DEAD Viability assay may be conducted based on automated fluorescent imaging with a tile-scanning confocal fluorescence microscope (Leica) and post imaging software analysis (ImageJ). Finally, immunocytochemistry may be performed with GFAP for 3D morphological analysis with Neurolucida software in Asteroid cocultures to assess whether Astroparticles induce astrogliosis, as determined by a change in cellular branching.

Synaptogenic proteins were expected to be present in the Astroparticles and not detected in Neurosomes or Plurisomes. One caveat with such an approach is that, since one is collecting membrane-bound proteins, it may not be possible to capture those that are secreted into the media. To address this, conditioned media can be collected and prepare Astroparticles from concentrated proteins, following established protocols. Even though similar reports of iAstro approaches have confirmed rapid production of synapse-promoting astrocytes by overexpression of Sox9 and NFIA, it is a possible caveat that the iAstros will not be able to produce synaptogenic proteins. Alternatively, one can determine the facility of iAstros at later time points of maturation, or traditional hPSC-derived astrocytes may be used for comparison. It is also expected that this protocol will generate reproducible characteristics between batches as observed in preliminary experiments. A caveat is that since this will be the first generation of Astroparticles, it is possible that one may encounter unexpected barriers such as aggregation, undesirable neutral charges or instability. As an alternative approach, the formulation (i.e., protein:lipid ratio, lipid type composition and method of Astroparticle production) may be modified as needed for each particular use or indication. Based on preliminary results, it is not expected that the disclosed Astroparticles will induce cellular toxicity at any of the doses indicated. Since a caveat with testing cellular response to Astroparticles is that the viability assays only indicate major cellular disruption, fluorescent oxidative stress may also be assayed as indicators of cellular damage, as previously demonstrated (6).

The premise that cell-specific proteins from hPSC-derived neural cells can be prepared and incorporated into nanovesicles is strong, and clearly feasible based on extant research expertise.

For each batch of Astroparticles, membrane proteins may be prepared from at least three separate rounds of differentiation of the iAstro hPSC line. For testing on cells, such preparations may be taken as biological replicates, on which multiple rounds of assays may be performed for technical replicates. Variability between each preparation may be reported, with Analysis of Variance statistical testing performed to assess for differences in production and toxicity. Groups may be blinded to the performing scientist with a coding scheme, and samples will not be de-identified until all data has been collected. Data from each batch of Astroparticles produced may also be compared to neurosomes, plurisomes, and/or liposomes for all assays.

### Example 6—Microfluidic-Based Synthesis of Neurosome Compositions

As noted above, a new class of biomimetic human neural NVs (a.k.a., “neurosomes” or “iAstrosomes”) have been developed and defined using a reproducible and scalable protein source from a pure population of hPSC-derived excitatory cortical neurons. Using a microfluidic-based synthesis method, proteolipids were bioengineered with these membrane proteins into NVs. Incorporation of neuron-derived membrane proteins did not affect the physicochemical properties of NVs and enhances their uptake into cultured neurons. Therapeutic efficacy of the disclosed neurosomes has been demonstrated with human neural sphere cultures, and also using the rodent nervous system assays, in vitro and in vivo, respectively. Finally, candidate adhesion proteins were identified in neurosomes which will pave the way for fully synthetic nanovesicle formulations. These studies advance the current paradigm of NV bioengineering for improved cellular targeting within the nervous system.

### Materials and Methods

### Cell Culture

Human pluripotent stem cells (hPSCs) (line WTC11, Coriell #GM25256) were cultured in pluripotent maintenance medium (hPSC medium) consisting of TeSR™-E8™ basal medium with supplements (STEMCELL Technologies) and 1× antibiotic-antimycotic (Gibco). Cells were infected with a lentivirus to express a membrane GFP transgene (Addgene #22479) and manually purified by clone selection. At 80% confluency, hPSCs were either collected for membrane protein extraction or differentiated to iNeurons.

Differentiation was prompted by exchanging basal hPSC medium with a neural-supportive medium (NM) consisting of DMEM/F-12 with GlutaMAX™ (Thermo Fisher), 0.5×N−2 and 0.5×B-27TM supplements (Gibco), 2 mg/mL heparin (Sigma-Aldrich), and 1× antibiotic-antimycotic, with the addition of 2 g/mL doxycycline hydrochloride (Dox; Sigma-Aldrich) for neuronal induction. Cells were maintained as a monolayer in NM+Dox for 2 days, treated with Accutase (Sigma-Aldrich) for cell detachment, and then replated on Matrigel-coated plates in the presence of rho-kinase inhibitor Y27632 (Tocris). On day 7, iNeurons were either collected for membrane protein extraction or treated with nanoparticles for association studies. Optionally, day 2 iNeurons were cultured in Aggrewell™ 800 24-well microwell plates (STEMCELL Technologies) to generate 3D neural spheres, as previously described.

### Membrane Protein Extraction and Quantification

Membrane proteins were extracted from iNeurons and hPSCs using a ProteoExtract® Native Membrane Protein Extraction Kit (Millipore Sigma) according to the manufacturer's protocol. Briefly, live cells were detached from tissue culture plates and transferred to 15 mL tubes. After centrifugation to remove supernatant, cell pellets were resuspended with 2 mL of PBS wash buffer, centrifuged again, and aspirated to remove PBS. Next, pellets were resuspended and washed twice with 2 mL of TE wash buffer provided with the kit. Pellets were resuspended in 10 L of protease inhibitor and 2 mL of extraction buffer 1 before incubation on ice for 10 min under gentle agitation. Samples were then centrifuged at 16000 μg for 15 min at 4° C. and the supernatant was then discarded. Pellets were resuspended in 5 μL of protease inhibitor and 1 mL of extraction buffer 2 before incubation on ice for 30 min under gentle agitation. Samples were then again centrifuged at 16000×g for 15 min at 4° C. The supernatant containing the extracted membrane proteins was transferred to another tube with 5 μL of protease inhibitor and stored at −80° C. until use.

Quantification of extracted membrane proteins was performed using a Pierce™ Rapid Gold BCA Protein Assay Kit (Fisher Scientific) according to the manufacturer's protocol. Briefly, albumin standards were prepared in 1×PBS at the following concentrations: 0, 25, 125, 250, 500, 750, 1000 and 1500 g/mL. Extracted membrane protein samples and extraction buffer 2 were diluted in 1×PBS (1:5, vol./vol.), with the latter serving as a blank.

The Rapid Gold BCA working reagent was prepared by mixing Reagent A with Reagent B (50:1, v/v). 20 μL of each sample (standards, membrane protein samples and extraction buffer 2 blank) were added in triplicate to a 96-well plate. 200 μL of the working reagent was then added to each well and incubated for 10 min at room temperature and no exposure to light. After the incubation, absorbance was measured at 480 nm on a FLUOstar® Omega microplate reader (BMG Labtech). Protein concentration was determined using a standard curve.

Nanovesicle synthesis and purification. NVs were synthesized using a NanoAssemblr (Precision Nanosystems). Formulation A consisted of Dipalmitoylphosphatidylcholine (DPPC), 1,2-dioleoyl-sn-glycero-3-phosphocholine (DOPC), and cholesterol (ovine wool, >98%) (4:3:3 molar ratio), while Formulation B was comprised of 16:0 1,2-dipalmitoyl-3-dimethylammonium-propane (DAP), DSPE-PEG2000, and cholesterol (4.2:1:4.8 molar ratio) (all from Avanti Polar Lipids, Inc).

1:100 (w/w) protein:lipid ratios were used for plurisome and neurosome formulations. The organic phase containing lipids was dissolved using 100% ethanol. The aqueous phase for Formulation A consisted of 1×PBS alone (for liposomes) or 1×PBS with extracted membrane proteins (for plurisome and neurosomes). The aqueous phase for Formulation B consisted of 125 mM sodium acetate buffer (pH=5.2) alone (for liposomes) or 125 mM sodium acetate buffer (pH=5.2) with extracted membrane proteins (for plurisome and neurosomes). After preparing the two phases for each formulation, the NanoAssemblr microfluidic chip was first washed with ethanol and then with either 1×PBS or 125 mM sodium acetate buffer (pH=5.2), depending on which formulation was to be prepared next. The organic and aqueous phases were loaded into individual syringes, allowed to warm for 3 min on a heating block set at 50° C. and then connected to the inlet ports of the chip. Particles were then synthesized using the following parameters for the machine: Formulation A−total flow rate=1 mL/min, organic flow rate=0.333 mL/min, aqueous flow rate=0.667 mL/min, waste_intial=0.15 mL, waste final=0.05 mL; Formulation B−total flow rate=1 mL/min, organic flow rate=0.350 mL/min, aqueous flow rate=0.650 mL/min, waste_intial=0.15 mL, waste final=0.05 mL. Synthesized particles were then dialyzed overnight using 1000 kDa Float-A-Lyzer™ G2 dialysis devices (Spectrum Labs) at 4° C. in 1×PBS (1:1000 v/v), with one buffer change after 1 hr. After dialysis, particles were collected and filtered using 0.22 m PVDF syringe filters (Fisher Scientific). Rhodamine labeled NVs were fabricated as described above with the addition of 0.005 mg of 1,2-dioleoyl-sn-glycero-3-phosphoethanolamine-N-(lissamine rhodamine B sulfonyl) (ammonium salt) to the organic phase for every 1 mM of lipids.

| Formulation | | Lipids | Molar |
|---|---|---|---|
| A | DPPC | Dipalmitoylphosphatidylcholine | 4 |
| | DOPC | 1,2-dioleoyl-sn-glycero-3-phosphocholine | 3 |
| | Cholesterol | | 3 |
| B | 16:0 DAP | 16:0 1,2-dipalmitoyl-3-dimethylammonium-propane | 4.2 |
| | DSPE-PEG2000 | ,2-distearoyl-sn-glycero-3-phosphoethanolamine N- | 1 |
| | | [carboxy(polyethylene glycol)-2000] | |
| | Cholesterol | | 4.8 |

### Membrane Protein Marker Detection

Presence of GFP and NCAM1 membrane proteins on the nanoparticles was verified using Western blot. After dialysis, nanoparticles were diluted in 1×PBS to a final volume of 1.5 mL and lipid concentration of 6 mM. 300 μL of nanoparticle samples were then centrifuged at 45000 rpm for 1 h at 4° C. The supernatant was then discarded, and the pellet was resuspended in 40 μL of 2.5% SDS buffer. All samples were denatured for 7 min at 95° C. After loading samples into the wells, the gel was run at 100V for 2 h before membrane transfer. Primary and secondary antibodies were then added to detect GFP and NCAM1. For GFP detection, Anti-GFP (Green Fluorescent Protein) Antibody (Chicken Antibodies, IgY Fraction) (GFP-1010) 1:2500 diluted (Aves Labs) followed by incubation with Goat anti-Chicken IgY H&L (HRP) (ab6877) 1:2000 diluted. For NCAM1 detection, Human/Mouse NCAM-1/CD56 Antibody (AF2408) 1:2500 diluted (R&D systems) followed by incubation with Anti-Goat IgG-HRP (HAF017) 1:2000 diluted. Gels were imaged using a Bio-Rad imaging system. Nanovesicle characterization: size, polydispersity index, zeta potential, and concentration

A Zetasizer system (Malvern Panalytical) was used to determine the size, polydispersity index (PDI) and zeta potential of all synthesized NVs. 500 μL of sample diluted 1:100 in 1×PBS was prepared in polystyrene cuvettes (Bio-Rad Laboratories) for size and PDI measurements. For each sample, a total of 3 runs with 10 measurements/run were performed; the average of these 3 runs was reported. For the zeta potential measurements, 10 μL of sample were diluted with 900 μL of MilliQ water and 90 μL of 1×PBS. Prepared samples were transferred to folded capillary disposable cuvettes (Malvern Panalytical). For each measurement, 3 runs with 15 measurements/run were performed; the average of these 3 runs was reported. A NanoSight NS300 system (Malvern Panalytical) was used to determine NV concentration after synthesis. Samples were prepared by diluting NVs in 1×PBS (1:10000) and loading onto the syringe pump. Acquisition settings were the following: Screen gain=1, camera level=13, flow ratio=1 mL/min and temperature=25° C. Five measurements of each sample were acquired for each sample, with a duration time of 60 sec/sample. A detection threshold of 7 was used to evaluate the final NV concentration.

### Nanovesicle Characterization: Cryo-EM

NV solutions were vitrified and imaged at the Baylor College of Medicine Cryo-Electron Microscopy Core Facility (Houston TX). Quantifoil R2/1, 200 Cu mesh Holey carbon grids were pretreated with air glow discharge for 45 sec in order to make the carbon surface hydrophilic. In addition, Quantifoil R2/1 200 Cu+4 nm thin carbon grids were also glow discharged for 10 s in order to test the efficacy of the added layer of continuous carbon with binding of the NVs. Vitrification was done using a Vitrobot Mark IV (FEI) operated at 8° C. with 100% humidity. 3 μL of NV sample was added to each grid, blotted for 1-3 s and immediately submerged in liquid ethane. Frozen grids were then transferred to a JEOL 3200FS microscope outfitted with a K2 Summit 4k×4k direct detector (Gatan) and a post-column energy filter set to 30 eV. To prevent any beam induced aberrations or astigmatisms, the microscope was carefully calibrated prior to image acquisition. Images were collected at magnifications of 15000× and 30000×, with pixel sizes of 2.392 and 1.232 angstroms, respectively. Images were collected using an exposure time of 1 s and an approximate dose rate of 20e-/Å²/s per image.

### Proteomic Analysis

Samples were sent to a mass spectrometry core facility for unbiased semi-quantitative proteomic analyses. Relative fraction of total (iFOT) values were determined (reported as log 10(iFOT×105), in order to perform two-tailed t-tests between normally distributed groups). Missing values (iFOT=0) were replaced with a value equal to one half of the minimum detected value in the dataset in order to determine fold change between groups (averaged across three replicates) using log 2 transformation.

### Metabolic Assays

For 2D toxicity assays, monolayers of memGFP-iNeurons were plated at a density of 60,000 cells/well in Matrigel-coated 96-well plates in NM+Dox. Cells were treated with NVs for 24-72 h at various concentrations ranging from 100 M to 1 mM, as indicated. At the time of the MTS assay, cells were incubated with Cell Titer 96® AQueous One Solution Reagent (Promega) in phenol red-free NM+Dox for 4 h according to the manufacturer's instructions. Absorbance was read at 490 nm using a plate reader (Tecan). 2D cellular viability was determined by subtracting blank values and normalizing to the control group without NVs. For 3D toxicity assays, iNeuron spheres were formed as indicated above and treated with NVs (100 or 500 M) for 24 h. Spheres were then incubated with equal volumes of CellTiter-Glo 3D Reagent (Promega) and phenol red-free NM+Dox in clear-bottom, opaque-walled 96-well plates. The assay was carried out according to the manufacturer's instructions and the luminescence (RLU) was determined using a plate reader (Tecan). Measured RLU values, which varied linearly with size of sphere, were normalized to the cross-sectional areas of each sphere. Results were then standardized to the mean value of the control groups (untreated, 0 M) for both formulations. Outliers were identified and removed using the ROUT method (based on maximum false discovery rate Q=1%) in GraphPad Prism.

### Fluorescence-Activated Cell Sorting (FACS)

FACS was performed as we previously elaborated with several modifications. Briefly, monolayers of iNeurons were plated at a density of 250,000 cells/well in Matrigel-coated 24-well plates in NM+Dox. On day 7, 100 M of rhodamine labeled NVs were added to neuron monolayers. Following a 24 h incubation, cells were gently detached with Accutase solution, washed with 1×PBS, centrifuged and washed again with 1×PBS. Cells were collected into flow cytometry tubes and run on BD LSRII flow cytometer using the Yel/Gm- 561 nm Laser and the PE filter 585/15 nm.

### Confocal Microscopy

iNeurons were plated on Matrigel-coated chamber slides for 48 h before treatment, then incubated with rhodamine-labeled NVs for 24 h. Cells were washed 3× in phosphate buffered saline (PBS) for 10 min each, then fixed with 4% paraformaldehyde for 30 min at 4° C., rinsed again with PBS 3× for 10 min each, and mounted with Fluoromount-G (Southern Biotech) on glass slides. After drying, slides were imaged on a DMi8 confocal microscope (Leica) with a 63× oil immersion objective to determine co-localization of rhodamine with cellular structures. 3D spheres were cleared in a solution of glycerol and fructose(43) before imaging with z-stack slides of X μm. Images were analyzed using ImageJ (National Institutes of Health).

### Reproducibility and Statistics

Significance was determined using two-tailed unpaired t-tests (for comparison between single conditions) or one-way analysis of variance (ANOVA) followed by post hoc multiple comparison test, as applicable. For 2D cellular viability, a 2-way ANOVA was performed, followed by Dunnett's multiple comparison test (comparing increasing NV concentrations to untreated cells within NV formulations A or B) or Tukey's multiple comparison test (comparing protein sources, within NV formulations A or B, across increasing concentrations). Formulations A and B were considered distinct and therefore not statistically compared in any experiments. Calculations were performed using the statistical analysis package in Prism software (GraphPad). Data from experiments using one biological replicate are averaged across technical replicates. For experiments with biological replicates, errors were calculated between replicates. Data are presented as mean±standard errors of the mean (SEM) unless otherwise indicated. In all figures, *=p≤0.05, **=p≤0.01, and ***=p≤0.001. Asterisks are not included if data were not acquired from biological replicates. The number of sampled units (n) are noted in figure captions.

### Results

### Synthesis and Characterization of Biomimetic Humanized Neural Nanovesicles

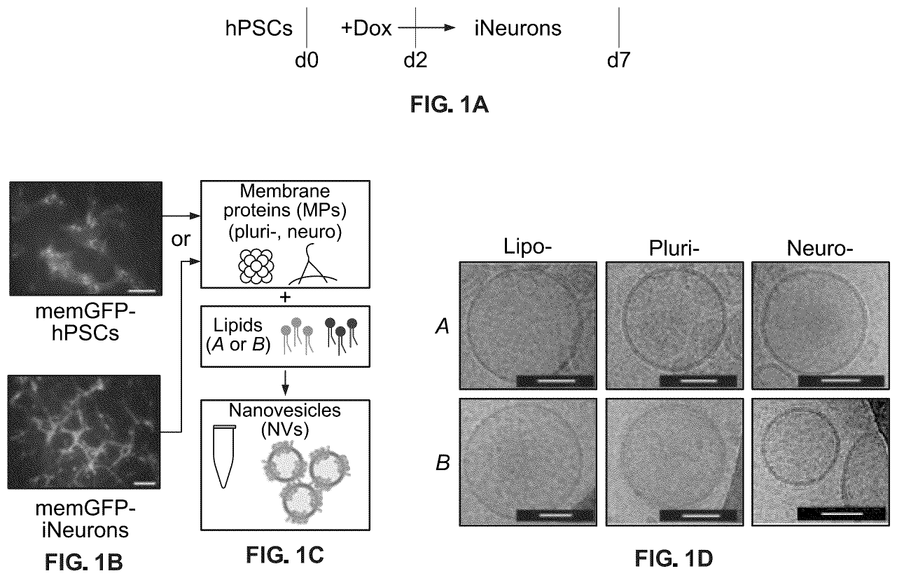

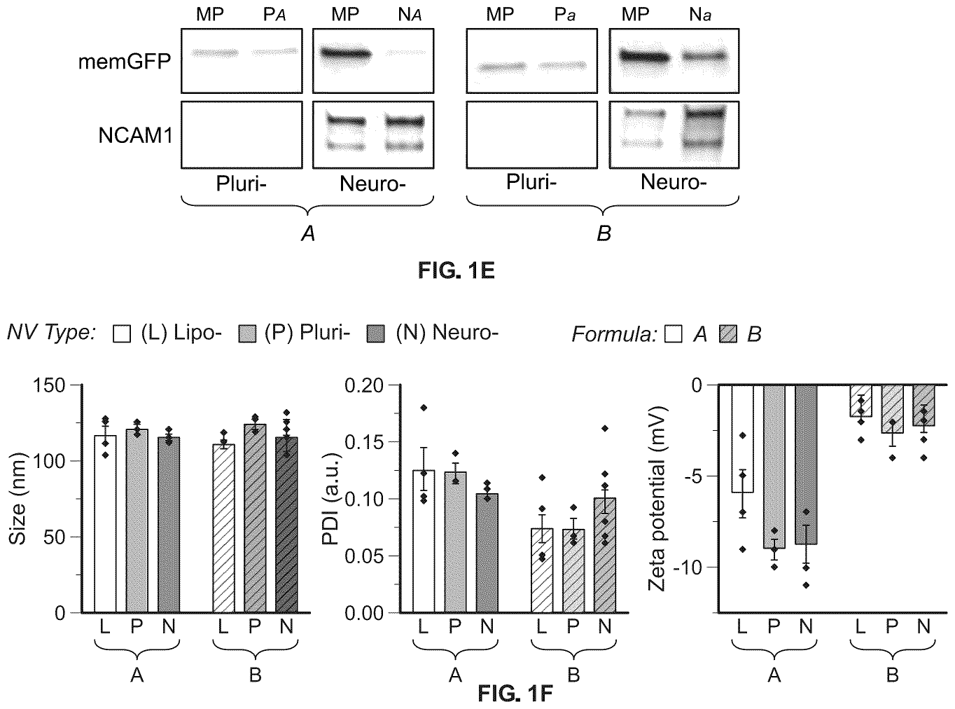

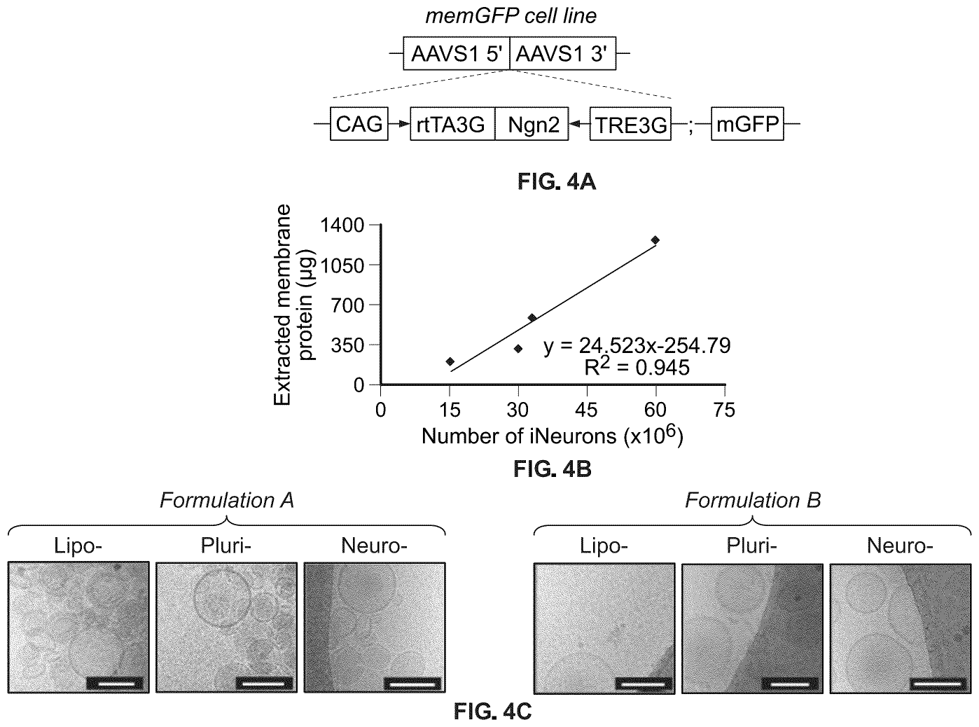

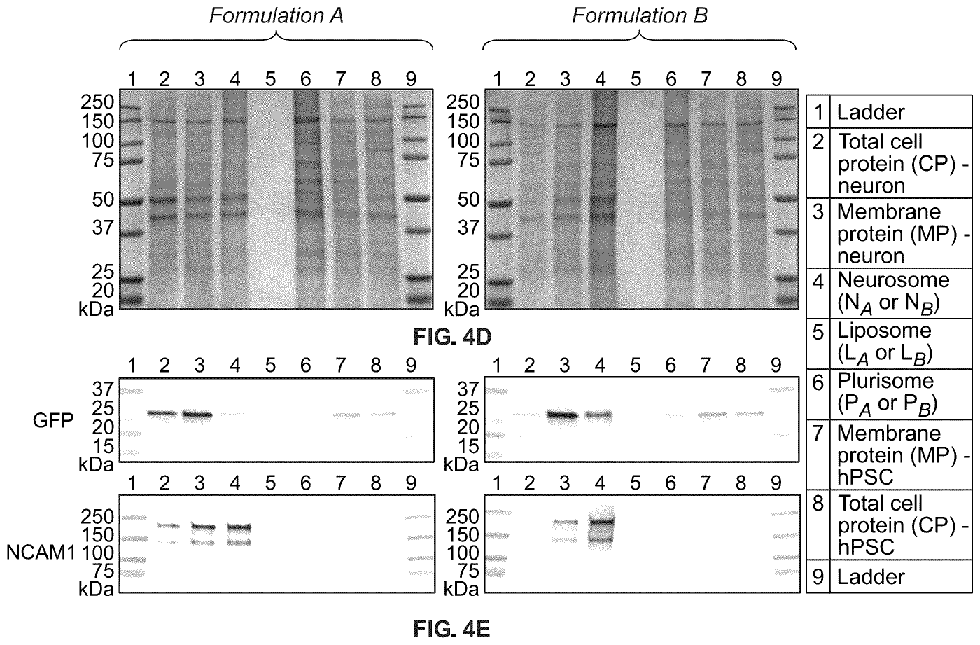

As a protein membrane source to functionalize NVs, a pure population of neurons were generated by directly inducing a genetically engineered hPSC line containing a doxycycline (dox)-inducible neurogenin 2 (ngn2) transgene as previously described in established protocols (FIG. 1A). Upon 7 days of induction in a neural-supportive medium, hPSCs were directly induced into a uniform population of excitatory cortical neurons (iNeurons) with distinctive neuronal morphology. Further, to determine whether bioengineered proteins can be produced in cells and transferred to NVs, a stable membrane-bound green fluorescent protein (memGFP) transgene was incorporated into hPSCs via lentiviral delivery, which continued expression during the differentiation process (FIG. 1B, FIG. 4A). Proteins from iNeurons and the parental hPSCs were then integrated into nanoparticle lipid bilayers to generate “neurosomes” (N) and “plurisomes” (P), respectively. As a negative control, we also prepared “liposomes” (L), i.e., NVs without incorporated membrane proteins.

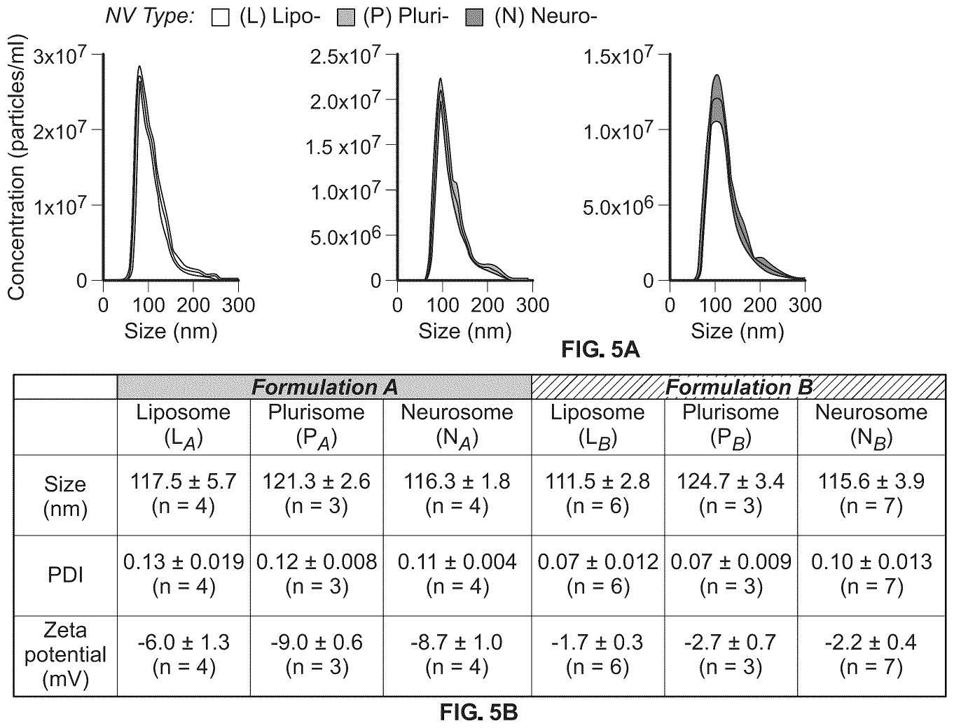

A previously optimized, modified microfluidic assembly protocol was used to generate biomimetic NVs with the NanoAssemblr™ system. In particular, cell-derived membrane proteins (FIG. 5B) were combined with phosphocholine-based phospholipids and cholesterol at a 1:100 (wt./wt.) ratio (FIG. 1C). Next, two distinct formulations were tested in order to enable the encapsulation of different cargo by varying the lipid content. Lipid formulation A (i.e., L_{A}, N_{A}, P_{A} for liposome formulation A, neurosome formulation A, and plurisome formulation A, respectively) was designed to deliver proteins or small hydrophobic or hydrophilic drugs, and consists of neutral lipids 1,2-dipalmitoyl-sn-glycero-3-phosphocholine (DPPC), 1,2-dioleoyl-sn-glycero-3-phosphocholine (DOPC), and cholesterol. Lipid formulation B was designed to deliver genetic cargo and consists of ionizable lipid 1,2-distearoyl-3-dimethylammonium-propane (16:0 DAP), 1,2-distearoyl-sn-glycero-3-phosphoethanolamine N-[carboxy(polyethylene glycol)-2000](DSPE-PEG), and cholesterol. After fabrication of the NVs, cryogenic transmission electron microscopy (cryo-TEM) visualization revealed preservation of a spherical bilayer structure and spherical shape irrespective of the alterations in the lipid formulation or cellular membrane protein source (FIG. 1D, FIG. 4C).

Following microfluidic synthesis, the transfer of membrane proteins into biomimetic NVs was tested. Coomassie blue staining after SDS-PAGE separation confirmed transfer of proteins (FIG. 4D). As evidenced by immunoblotting, we observed the specific presence of transgene-derived memGFP only in NVs integrated with cell-derived proteins (FIG. 1E, FIG. 4E). Subsequently, NCAM1 was used as an additional indicator of endogenous cell type-specific protein carry-over as it is a known neural-restricted protein. NCAM1 was present in protein fractions of neurosomes originating from iNeurons (e.g., N_{A} and N_{B}), but not plurisomes originating from hPSCs (e.g., P_{A} and P_{B}), as expected. The same trend was observed among both lipid formulations A and B. Hence, the transfer of both bioengineered membrane proteins as well as endogenous proteins into NVs was confirmed using human cells.

To determine whether the incorporation of human proteins would disrupt defining features of NVs, we next characterized the physicochemical properties and reproducibility of distinct formulations and protein sources. First, we quantified NV concentration using a NanoSight system (FIG. 5A) and verified homogeneity of size, polydispersity index (PDI), and surface charge (zeta potential) using a Zetasizer Nano ZS system (FIG. 1F, FIG. 5B), between and among formulations. Neither the mean diameter nor the PDI was significantly altered by modification of membrane protein content (i.e., liposomes versus neurosomes versus plurisomes) and the lipid formulation (i.e., A versus B). However, fundamental differences in lipid composition between formulations A and B produced significant differences in zeta potential between formulations A and B, but not among groups of the same formulation. More specifically, liposomes, plurisomes, and neurosomes in formulation B demonstrated average decreases of 3.7-fold, 3.4-fold, and 3.9-fold, respectively, compared to formulation A Overall, these characterizations verified the ability to reliably produce uniform NVs using these formulations.

### Confirmation of Cellular Association of NVs

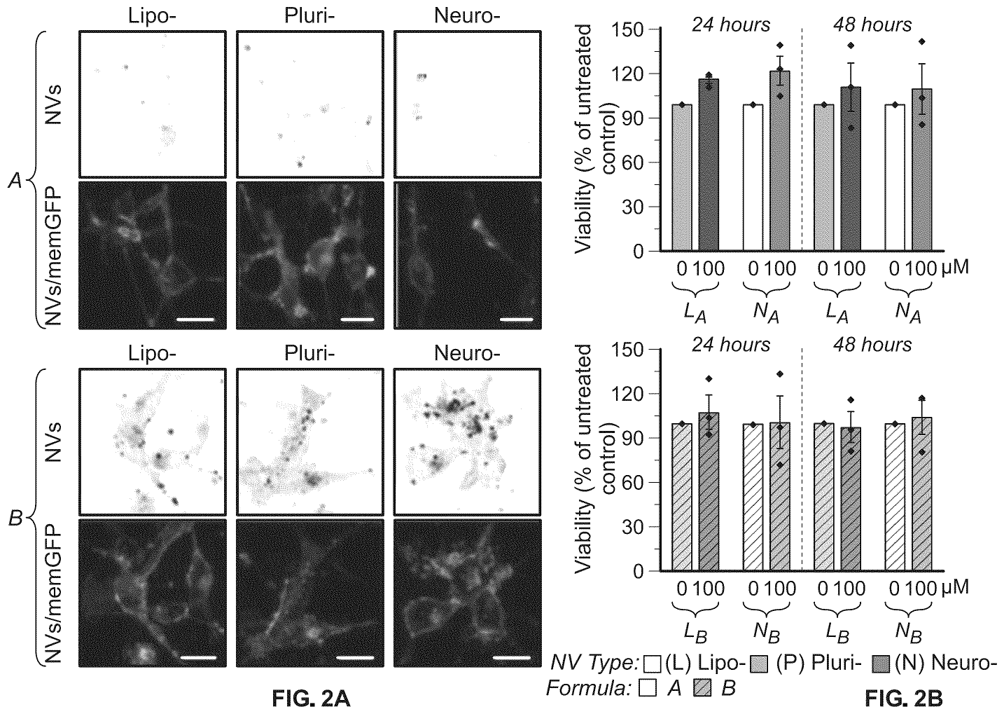

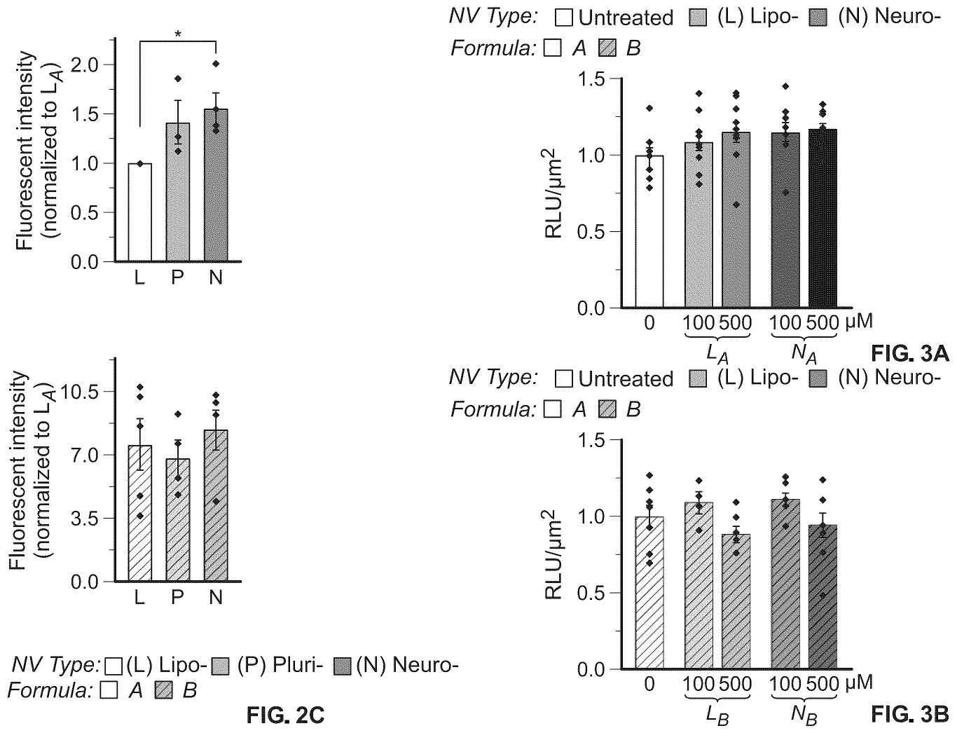

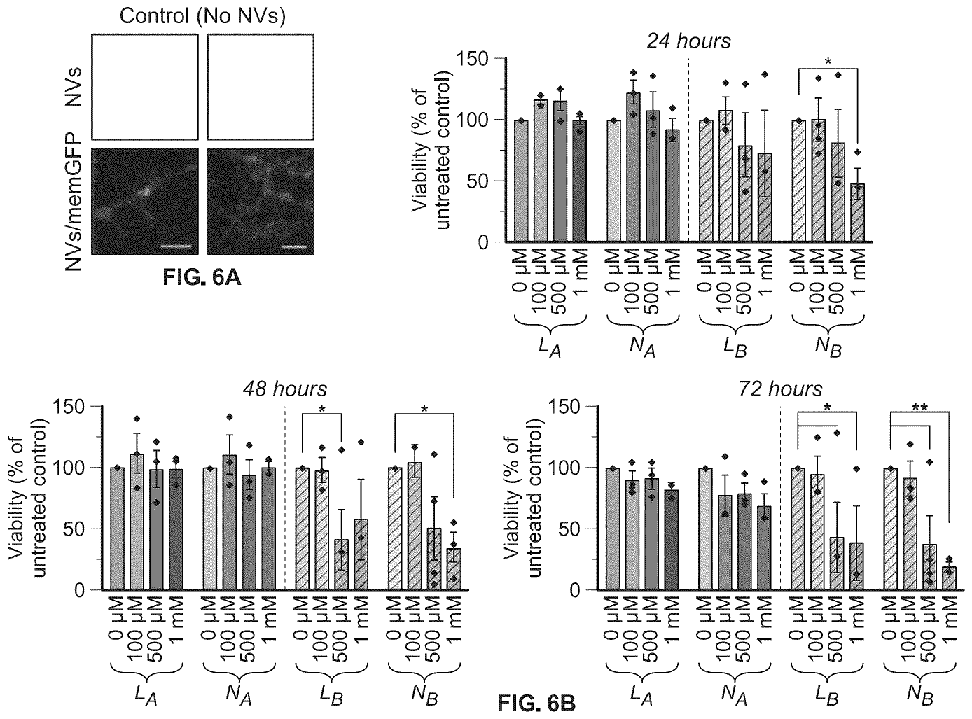

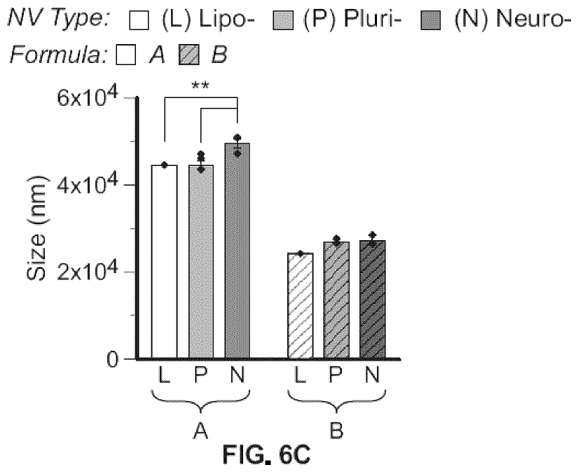

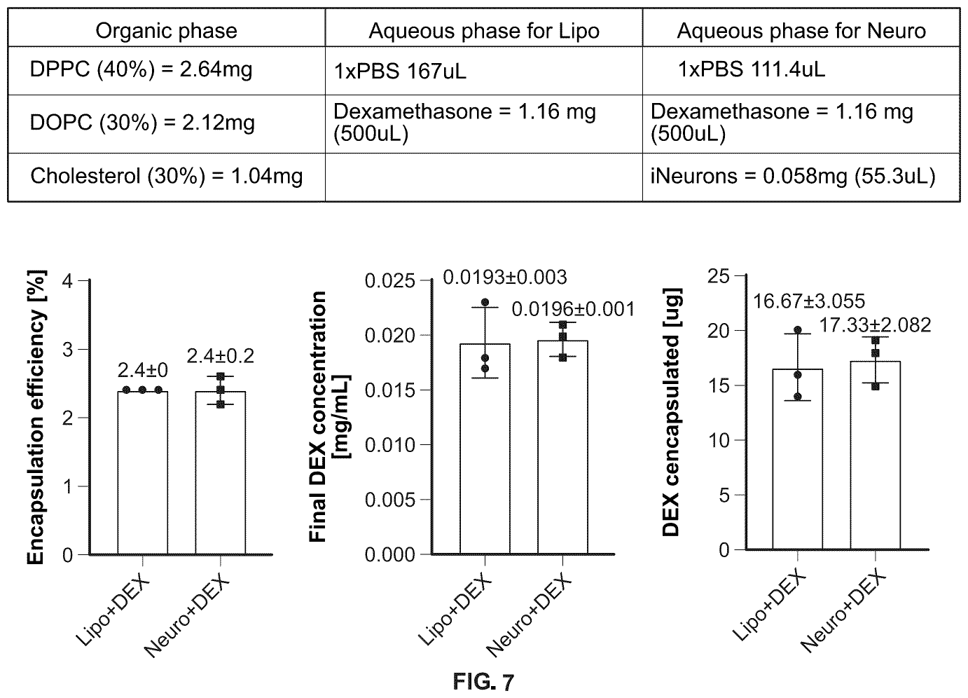

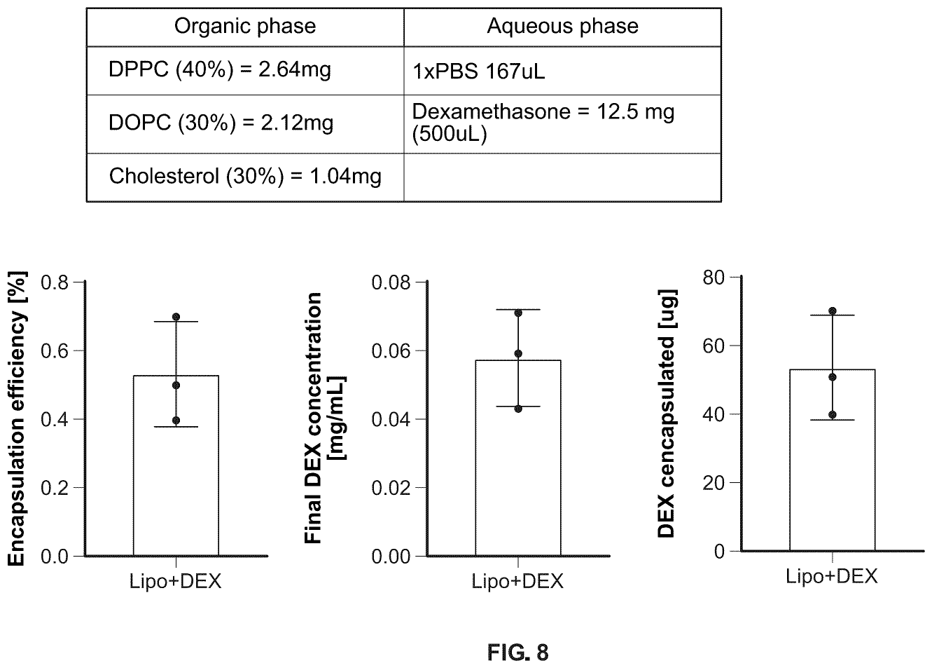

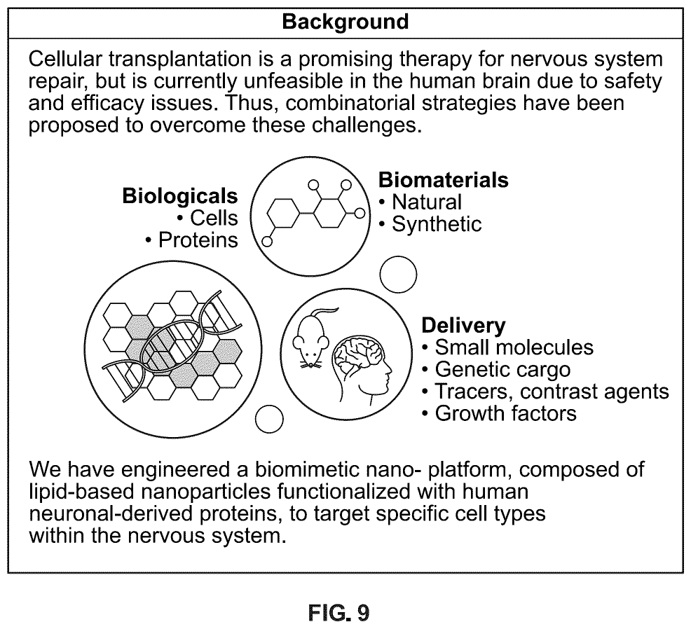

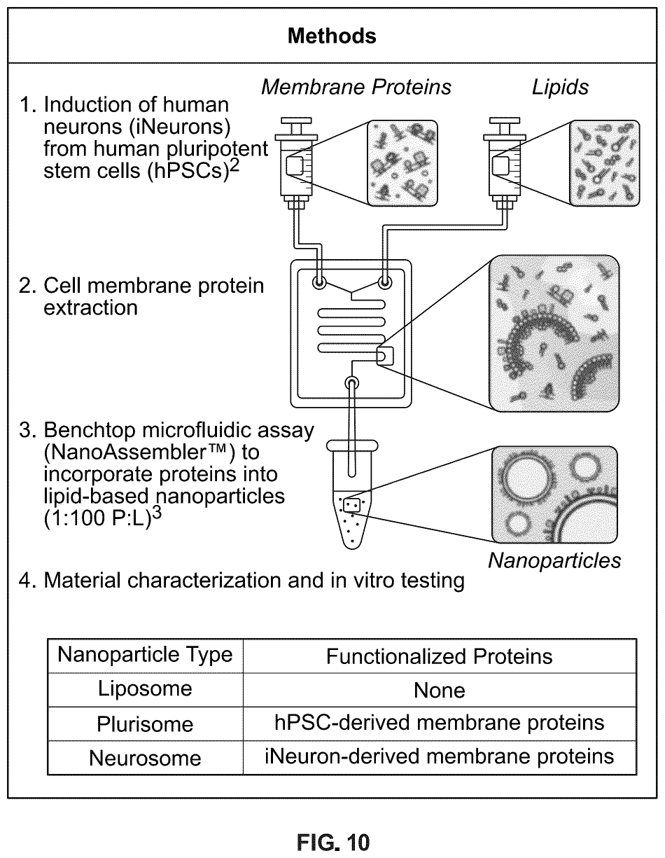

NVs were labeled for 24 hr before thorough washing to remove all non-associated and non-internalized NVs. First, uptake was qualitatively confirmed with confocal microscopy (FIG. 2A, FIG. 6A), wherein it was observed that colocalization of cellular memGFP and rhodamine nanoparticles was increased in formulation B compared to formulation A Notably, within formulation B, we detected a gradient pattern of nanoparticle association, with higher rhodamine signal in N_{B} than P_{B}, which was subsequently higher than L_{B}.

Next, we established treatment concentrations that did not inadvertently lead to cellular toxicity during measurement. memGFP-iNeurons were cultured in monolayers and incubated with NVs for 24-72 hrs at concentrations ranging from 100 M to 1 mM. Neither lipid formulation of neurosomes or liposomes resulted in significant cytotoxicity at 100 M up to 48 hrs of treatment time compared to untreated cells, as determined by a viability assay (FIG. 2B). However, an increase in NV concentration surpassing 500 M in formulation B led to decreased viability over time; thus, 24-hour treatment of 100 M NVs was determined as the upper limit of cellular tolerance for further experimentation (FIG. 6B).

Quantitative evaluation of iNeuron-NV association was assessed using high throughput fluorescence-activated cell sorting (FACS), wherein iNeuron monolayers were incubated with 100 M rhodamine-labeled NVs for 24 hrs before gentle dissociation and sorting. Untreated cells without NVs were used to set the gate. Fluorescent intensity was normalized to the control liposome group from lipid formulation A (L_{A}) to account for variance in NV intensity among groups (FIG. 6C). Again, iNeurons exhibited higher preferential association with plurisomes (1.42±0.22-fold increase in fluorescent intensity of P_{A} compared to L_{A}) and significantly higher preferential association with neurosomes (1.44±0.16-fold increase in fluorescent intensity of N_{A} compared to L_{A}; p=0.014) compared to liposomes from formulation A (FIG. 2C). Cellular association was observed to be higher overall with NVs generated with lipid formulation B (7.55±1.45-, 6.78±1.02-, and 8.35±1.06-fold increases in fluorescent intensity of L_{B}, P_{B}, and N_{B} compared to L_{A}), although there were no significant trends between NVs of different protein sources.

Finally, to assess biodistribution dynamics within intact brain, rhodamine-labeled NVs were injected in the cortex of postnatal day-2 immunodeficient (NSG) mice (liposomes in the left hemisphere and neurosome in right hemisphere of the same animal). Live imaging (IVIS Spectrum) confirmed brain targeting and retention of NVs for at least 48 hrs. This confirms potential for pre-clinical utilization of NVs to delivery neuroprotective or neuromodulatory payloads into cells of the nervous system.

Here, a multifunctional, biomimetic nanotechnology platform is described and validated that is promising for targeting neural cell types and delivering therapeutic cargo. Two well-studied lipid backbones were used for the fabrication of biomimetic NVs. Each of these formulations demonstrated efficacy on encapsulating and delivering different therapeutic cargos: formulation A for hydrophilic and hydrophobic agents; formulation B for genetic cargos such as siRNA or mRNA. Subsequently, we verified incorporation of endogenous neural specific proteins (e.g., NCAM1) and a representative exogenous membrane protein (e.g., memGFP) into the NVs surface when using a microfluidic-based synthesis method. We then characterized both the physical and biomimetic properties of the NVs, including size, surface charge, polydispersity, morphology, and integration of cell-derived membrane proteins toxicity. Next, after investigating an upper limit of cellular tolerance for in vitro studies, it was shown that neurosomes generated from Formulation A exhibit an increased capacity to accumulate in hPSC-derived neurons in vitro using both qualitative and quantitative analysis methods. Finally, an innovative 3D human organoid-like neural testing platform was utilized to assess the non-toxicity of the nanoparticles. The NVs biocompatibility and brain tissue retention time was assessed using in vivo imaging system for 48 hr.

With a stable protein source and reproducible fabrication method in hand, characterization of the synthesized NVs corroborated the successful maintenance of key physiochemical and biological properties following synthesis. In particular, the inclusion of membrane proteins in this fabrication process did not negatively impact the formation, size and homogeneity of the resulting bilayered NVs, especially when compared to their non-protein containing counterparts—liposomes. Furthermore, the detection of both endogenous and exogenous membrane proteins on the synthesized NVs validated the successful carry-over of desired proteins from the cell source. Lastly, the consistency of these results across multiple batches of NVs and the use of microfluidic fabrication method that allow scale up through process parallelization demonstrated the reproducibility of this biomimetic platform.

In contrast to the current state-of-the-art for nanoparticle production (complex bottom-up approaches that rely on the assembling of individual building blocks for synthesis before further surface functionalization and purification), the disclosed high-yield, scalable, and reproducible top-down method takes advantage of a microfluidic assembly platform to reproduce the complexity of neural cellular membranes on nanoparticle surfaces. This cost-effective, standardizable one-step process does not require chemical synthesis or solvent purification. Furthermore, this bioengineered approach allows for testing of various protein-lipid formulations to enhance cellular targeting and association. The use of transgenic hPSC-derived inducible neurons (iNeurons) provides a synchronous, pure population of cells as a reproducible and unlimited source of human neuronal membrane proteins within a matter of days. These customizable biomimetic strategies represent a paradigm shift in the design and engineering of neural-specific nanoparticles, enabling next-generation technology platforms capable of effectively interfacing and interacting with complex biological systems.

Following the neurosomes' higher neural association, we believe this study provides valuable information that could be relevant for future studies involving the delivery of therapies directed at the treatment of neural diseases. One example can be the delivery of neural growth factor after an injury. Furthermore, we expect neurosomes can be rapidly translated to clinical applications as they can be manufactured using similar, standardized manufacturing protocols (scalability, good manufacturing practices) previously established for liposomes while using iNeurons as a source for the human neuronal membrane proteins. Given the translational advantages and the fabrication tunability (i.e., both lipids and proteins) offered by this technology, these biomimetic NVs will provide an innovative approach for the targeted delivery of needed therapeutic cargoes to neurological diseases.

### REFERENCES

The following references, to the extent that they provide exemplary procedural or other details supplementary to those set forth herein, are specifically incorporated herein by reference:
ALLEN, NJ and LYONS, DA, “Glia as architects of central nervous system formation and function,” Science, 362(6411):181-185 (October 2018).
AL V AREZ-ERVITI, L et al., “Delivery of siRNA to the mouse brain by systemic injection of targeted exosomes,” Nat. Biotechnol., 29(4):341-345 (March 2011).
AMIR, R E et al., “Rett syndrome is caused by mutations in X-linked MECP2, encoding methyl-CpG-binding protein 2,” Nat. Genet., 23(2):185-188 (October 1999).
AMIT, M et al., “Loss of p53 drives neuron reprogramming in head and neck cancer,” Nature, 578(7795):449-454 (February 2020).
BARBAR, L et al., “CD49f is a novel marker of functional and reactive human iPSC-derived astrocytes,” Neuron., 107(3):436-453 (August 2020).
BA YRAKTAR, O A et al., “Astrocyte layers in the mammalian cerebral cortex revealed by a single-cell in situ transcriptomic map,” Nat. Neurosci., 23(4):500-509 (March 2020).
BEYER, F et al., “Do neural stem cells have a choice?heterogenic outcome of cell fate acquisition in different injury models,” Int. JMal. Sci., 20(2):455 (January 2019).
BOADA, C et al., “Rapamycin-loaded leukosomes reverse vascular inflammation,” Circ. Res., 126(1):25-37 (October 2019).
CHAHROUR, M and ZOGHBI, HY, “The story of Rett syndrome: from clinic to neurobiology,” Neuron, 56(3):422-437 (November 2007).
CORBO, C et al., “Unveiling the in vivo protein corona of circulating leukocyte-like carriers,” ACS Nano., 11(3):3262-3273 (March 2017).
CVETKOVIC, C et al., “Synaptic microcircuit modeling with 3D cocultures of astrocytes and neurons from human pluripotent stem cells,” J Vis. Exp., 138:58034 (August 2018).
DALLERAC, G et al., “Versatile control of synaptic circuits by astrocytes: where, when and how,” Nat. Rev. Neurosci., 19(12):729-743 (December 2018).
DANTE, S et al., “Selective targeting of neurons with inorganic nanoparticles: revealing the crucial role of nanoparticle surface charge,” ACS Nano., 11(7):6630-6640 (June 2017).
DAY A, S and BERNS, KI, “Gene therapy using adeno-associated virus vectors,” Clin. Microbial. Rev., 21(4):583-593 (October 2008).
DEKKERS, J F et al., “High-resolution 3D imaging of fixed and cleared organoids,” Nat. Protoc., 14: 1756-1771 (May 2019).
DINET, V et al., “Brain-immune interactions and neuroinflammation after traumatic brain injury,” Front. Neurosci., 13: 1178 (November 2019).
ELOY, J O et al., “Liposomes as carriers of hydrophilic small molecule drugs: strategies to enhance encapsulation and delivery,” Colloids Surf B. Biointerfaces, 123:345-363 (September 2014).
EVANGELOPOULOS, M et al., “Trends towards biomimicry in theranostics,” Nanomaterials (Basel), 8(9):637 (August 2018).
FANG, R H et al., “Cell membrane-derived nanomaterials for biomedical applications,” Biomaterials, 128:69-83 (March 2017).
FARHY-TSELNICKER, I, and ALLEN, NJ, “Astrocytes, neurons, synapses: a tripartite view on cortical circuit development,” Neural Dev., 13(1):7 (May 2018).
FAVUZZI, E et al., “Activity-dependent gating of parvalbumin interneuron function by the perineuronal net protein brevican,” Neuron., 95(3):639-655 (July 2017).
FOO, L C et al., “Development of a method for the purification and culture of rodent astrocytes,” Neuron., 71(5):799-811 (September 2011).
FU, W Y et al., “Targeting neuroinflammation as a therapeutic strategy for alzheimer's disease: Mechanisms, drug candidates, and new opportunities,” ACS Chem. Neurosci., 10(2):872-879 (September 2018).
GENC, R et al., “Green synthesis of gold nanoparticles using glycerol-incorporated nanosized liposomes,” Langmuir., 27(17): 10894-10900 (August 2011).
GRABRUCKER, A M et al., “Development of novel Zn²⁺ loaded nanoparticles designed for cell-type targeted drug release in CNS neurons: in vitro evidences,” PLoS One, 6(3):e17851 (March 2011).
GREENHALGH, A D et al., “Immune cell regulation of glia during CNS injury and disease,” Nat. Rev. Neurosci., 21(3): 139-152 (February 2020).
GUZMAN-MARTINEZ, L et al., “Neuroinflammation as a common feature of neurodegenerative disorders,” Front. Pharmacol., 10:1008 (September 2019).
HA, D et al., “Exosomes as therapeutic drug carriers and delivery vehicles across biological membranes: Current perspectives and future challenges,” Acta. Pharm. Sin. B., 6(4):287-296 (March 2016).
HAMPEL, H et al., “A path Toward Precision Medicine for Neuroinflammatory Mechanisms in Alzheimer's Disease,” Front Immunol., 11:456 (March 2020).
HILLEN, AEJ et al., “Cell adhesion and matricellular support by astrocytes of the tripartite synapse,” Prog. Neurobiol., 165-167:66-86 (February 2018).
HOFFMAN, J R et al., “Microfluidic Assembly of Liposomes with Tunable Size and Coloading Capabilities,” Methods Mol. Biol., 1792:205-214 (2018).
INGAWALE, DK and MANDLIK, SK, “New insights into the novel anti-inflammatory mode of action of glucocorticoids,” Immunopharmacol. Immunotoxicol., 42(2):59-73 (February 2020).
JANG, S C et al., “Bioinspired exosome-mimetic nanovesicles for targeted delivery of chemotherapeutics to malignant tumors,” ACS Nano., 7(9):7698-7710 (September 2013).
KAMELSKA-SADOWSKA, A M et al., “Review of the current knowledge on the role of stem cell transplantation in neurorehabilitation,” Biomed Res. Int., 2019:3290894 (February 2019).
KASSI, AAY et al., “Enduring neuroprotective effect of subacute neural stem cell transplantation after penetrating TBI,” Front. Neural., 9: 1097 (January 2019).
KHAKH, BS and SOFRONIEW, MV, “Diversity of astrocyte functions and phenotypes in neural circuits,” Nat. Neurosci., 18(7):942-952 (July 2015).
KLEIN, R S et al., “Neuroinflammation during RNA viral infections,” Annu. Rev. Immunol., 37:73-95 (April 2019).
KRENCIK, R et al., “Dysregulation of astrocyte extracellular signaling in Costello syndrome,” Sci. Transl. Med, 7(286):286ra66 (May 2015).
KRENCIK, R et al., “Specification of transplantable astroglial subtypes from human pluripotent stem cells,” Nat, Biotechnol., 29(6):528-534 (May 2011).
KRENCIK, R et al., “Systematic three-dimensional coculture rapidly recapitulates interactions between human neurons and astrocytes,” Stem Cell Reports, 9(6):1745-1753 (November 2017).
LEE, E and CHUNG, WS, “Glial control of synapse number in healthy and diseased brain,” Front. Cell. Neurosci., 13:42 (February 2019).
LI, X et al., “Fast Generation of Functional Subtype Astrocytes from Human Pluripotent Stem Cells,” Stem Cell Reports, 11(4):998-1008 (September 2018).
LIDDELOW, SA and BARRES, BA, “Reactive Astrocytes: Production, Function, and Therapeutic Potential,” Immunity, 46(6):957-967 (June 2017).
LIU, Y et al., “Medial ganglionic eminence-like cells derived from human embryonic stem cells correct learning and memory deficits,” Nat. Biotechnol., 31(5):440-447 (April 2013).
LIU, Z et al., “Looking into the future: toward advanced 3D biomaterials for stem-cell-based regenerative medicine,” Adv. Mater., 30(17):e1705388 (February 2018).
LUK, BT and ZHANG, L, “Cell membrane-camouflaged nanoparticles for drug delivery,” J Control. Rel., 220(Pt B):600-607 (July 2015).
MARTINEZ, J O et al., “Biomimetic nanoparticles with enhanced affinity towards activated endothelium as versatile tools for theranostic drug delivery,” Theranostics, 8(4):1131-1145 (January 2018).
MCQUADE, A et al., “Development and validation of a simplified method to generate human microglia from pluripotent stem cells,” Mol. Neurodegener., 13(1):67 (December 2018).
MOLINARO, R et al., “Biomimetic proteolipid vesicles for targeting inflamed tissues,” Nat. Mater, 15(9): 1037-1046 (May 2016).
MOLINARO, R et al., “Design and development of biomimetic nanovesicles using a microfluidic approach,” Adv. Mater., 30(15):e1702749 (March 2018).
MOLINARO, R et al., “Macrophage-derived nanovesicles exert intrinsic anti-inflammatory properties and prolong survival in sepsis through a direct interaction with macrophages,” Nanoscale., 11(28): 13576-13586 (July 2019).
MOLINARO, R et al., “Vascular inflammation: a novel access route for nanomedicine,” Methodist DeBakey Cardiovasc. J, 12(3): 169-17 4 (September 2016).
MOLOFSKY, A V et al., “Astrocytes and disease: a neurodevelopmental perspective,” Genes Dev., 26(9):891-907 (May 2012).
MORIARTY, N et al., “Primary tissue for cellular brain repair in Parkinson's disease: Promise, problems and the potential of biomaterials,” Eur J Neurosci., 49(4):472-486 (July 2018).
NASR, I W et al., “Neuroimmune responses in the developing brain following traumatic brain injury,” Exp. Neural., 320:112957 (May 2019).
NEEDHAM, E J et al., “The immunological response to traumatic brain injury,” J Neuroimmunol., 332: 112-125 (April 2019).
OBERHEIM BUSH, NA and NEDERGAARD, M, “Do evolutionary changes in astrocytes contribute to the computational power of the hominid brain,” Neurochem. Res., 42(9):2577-2587 (August 2017).
PALOMBA, R et al., “Biomimetic carriers mimicking leukocyte plasma membrane to increase tumor vasculature permeability,” Sci. Rep., 6:34422 (October 2016).
PARODI, A et al., “Synthetic nanoparticles functionalized with biomimetic leukocyte membranes possess cell-like functions,” Nat. Nanotechnol., 8(1):61-68 (December 2012).
PATEL, R et al., “Concepts toward directing human astroplasticity to promote neuroregeneration,” Dev. Dyn., 248(1):21-33 (September 2018).
POLANCO, J C et al., “Exosomes taken up by neurons hijack the endosomal pathway to spread to interconnected neurons,” Acta. Neuropathol. Commun., 6(1):10 (February 2018).
RISHER, W C et al., “Thrombospondin receptor a28-1 promotes synaptogenesis and spinogenesis via postsynaptic Rael,” J Cell. Biol., 217(10):3747-3765 (July 2018).
SAEEDI, S et al., “The emerging role of exosomes in mental disorders,” Transl. Psych., 9(1): 122 (March 2019).
SEMPLE, S C et al., “Efficient encapsulation of antisense oligonucleotides in lipid vesicles using ionizable aminolipids: formation of novel small multilamellar vesicle structures,” Biochim. Biophys. Acta., 1510(1-2):152-166 (February 2001).
SIMAO, D et al., “Recapitulation of human neural microenvironment signatures in iPSC-derived NPC 3D differentiation,” Stem Cell Reports, 11(2):552-564 (August 2018).
SINGH, S K et al., “Astrocytes assemble thalamocortical synapses by bridging NRX1α and NL1 via Hevin,” Cell, 164(1-2): 183-196 (January 2016).
SONG, I and DITYATEV, A, “Crosstalk between glia, extracellular matrix and neurons,” Brain Res. Bull., 136: 101-108 (March 2017).
SOSUNOV, A A et al., “Phenotypic heterogeneity and plasticity of isocortical and hippocampal astrocytes in the human brain,” J Neurosci., 34(6):2285-2298 (February 2014).
TCHIEU, J et al., “NFIA is a gliogenic switch enabling rapid derivation of functional human astrocytes from pluripotent stem cells,” Nat. Biotechnol., 37(3):267-275 (February 2019).
THERY, C et al., “Exosomes: composition, biogenesis and function,” Nat. Rev. Immunol., 2(8):569-579 (August 2002).
VAINCHTEIN, ID and MOLOFSKY, AV, “Astrocytes and microglia: In “Sickness and in Health,” Trends Neurosci., 43(3):144-154 (February 2020).
VALENTE, P et al., “Conopeptide-Functionalized Nanoparticles Selectively Antagonize Extrasynaptic N-Methyl-d-aspartate Receptors and Protect Hippocampal Neurons from Excitotoxicity In vitro,” ACS Nano., 14(6):6866-6877 (June 2020).
VOET, S et al., “Inflammasomes in neuroinflammatory and neurodegenerative diseases,” EMBO Mal. Med, 11(6):e10248 (April 2019).
WU, S et al., “On the viability and potential value of stem cells for repair and treatment of central neurotrauma: overview and speculations,” Front. Neural., 9:602 (August 2018).
XIA, J et al., “The role of adhesions between homologous cancer cells in tumor progression and targeted therapy,” Expert Rev. Anticancer Ther., 17(6):517-526 (May 2017).
XU, S et al., “Glial cells: role of the immune response in ischemic stroke,” Front. Immunol., 11:294 (February 2020).
YU, X et al., “Improved tools to study astrocytes,” Nat. Rev. Neurosci., 21(3):121-138 (February 2020).
ZHANG, F et al., “Multimodal fast optical interrogation of neural circuitry,” Nature, 446(7136):633-639 (April 2007).
ZHANG, Y et al., “Purification and characterization of progenitor and mature human astrocytes reveals transcriptional and functional differences with mouse,” Neuron., 89(1):37-53 (December 2015).
ZHU, J Y et al., “Preferential cancer cell self-recognition and tumor self-targeting by coating nanoparticles with homotypic cancer cell membranes,” Nano Lett., 16(9):5895-5901 (August 2016).
ZIBARA, K et al., “Combination of drug and stem cells neurotherapy: Potential interventions in neurotrauma and traumatic brain injury,” Neuropharmacology, 145(Pt B): 177-198 (September 2018).
ZINGER, A et al., “Proteolytic nanoparticles replace a surgical blade by controllably remodeling the oral connective tissue,” ACS Nano., 12(2): 1482-1490 (January 2018).
ZINGER, A et. al., “Collagenase nanoparticles enhance the penetration of drugs into pancreatic tumors,” ACS Nano., 13(10): 11008-11021 (September 2019).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are specifically incorporated herein by reference to the same extent as if each reference was individually and specifically indicated to be incorporated by reference, and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing,” with reference to an element or elements is intended to provide support for a similar aspect or embodiment of the invention that “consists of,” “consists essentially of,” or “substantially comprises” the particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and methods and in the steps or in the sequence of steps of the method described herein without departing from the concept, spirit and scope of the invention. More specifically, it will be apparent that certain agents that are chemically- or physiologically-related may be substituted for the agents described herein while the same or similar results would be achieved. All such similar substitutes and modifications apparent to those ordinarily skilled in the art are deemed to be within the spirit, scope, and concept of the invention as defined by the appended claims.

What is claimed is:

1. A drug delivery composition comprising a population of biomimetic proteolipid nanovesicles composed of synthetic lipids, phospholipids, and/or cholesterol, enriched of one or more neuronal-derived membrane fragments, polypeptides, or biologically-active fragments or derivatives thereof, and surrounding an aqueous core.

2. The drug delivery composition of claim 1, wherein the proteolipid nanovesicles comprise at least one astrocyte-derived protein or active fragment thereof, at least one neuronal-derived protein or active fragment thereof, or a combination thereof on their surface.

3. The drug delivery composition of claim 1, further comprising at least one therapeutic agent, including, for example, a therapeutic drug, a chemotherapeutic, an antiangiogenic compound, an antibiotic, an analgesic, an anti-inflammatory agent, a neuromodulatory agent, a neurogenic protein, polypeptide, or peptide, such as MeCP2, a DNA molecule, an RNA molecule, an antibody, or any combination thereof.

4. The drug delivery composition of claim 3, wherein the therapeutic agent is derived from a population of human pluripotent stem cells or a population of neurons isolated or derived from one or more human pluripotent stem cells (hPSC).

5. The drug delivery composition of claim 1, further comprising at least one therapeutic agent selected from the group consisting of an immune-stimulating agent, a tumor growth inhibitor, a protein, a peptide, an RNA molecule, a DNA molecule, an siRNA molecule, a RNAi molecule, a ssRNA molecule, a growth factor, an enzyme inhibitor, a binding protein, a blocking peptide, and any combination thereof.

6. The drug delivery composition of claim 1, wherein the proteolipid nanovesicles are adapted configured to release the at least one therapeutic agent in response to an external stimulus, in response to a change in the environment of the population of biomimetic proteolipid nanovesicles, or as a result of degradation of a population of astroparticles, neurosomes, plurisomes, or any combination thereof.

7. The drug delivery composition of claim 1, wherein degradation of the population of biomimetic proteolipid nanovesicles, astroparticles, neurosomes, or plurisomes occurs via enzyme-facilitated biodegradation of one or more of the lipids, the phospholipids, or the cholesterol comprising them.

8. The drug delivery composition of claim 1, wherein the neuron-derived membrane fragments comprise at least one cellular-targeting moiety.

9. The drug delivery composition of claim 8, wherein the at least one cellular-targeting moiety is a neuronal cell-targeting moiety, a ligand, a ligand-binding moiety, a receptor, such as ICAM1 and L1CAM, a receptor-binding moiety, an antibody or an antigen-binding fragment thereof, and any combination thereof.

10. The drug delivery composition of claim 9, wherein the at least a first cellular-targeting moiety comprises a plurality of distinct antigenic ligands that elicit one or more target-specific immune responses in a mammalian host cell that is contacted with the population of nanovesicles.

11. The drug delivery composition of claim 1, further comprising a diagnostic agent or a theranostic agent, or a combination thereof.

12. The drug delivery composition of claim 1, comprising a diagnostic reagent selected from the group consisting of an imaging agent, a contrast agent, a fluorescent label, a radiolabel, a magnetic resonance imaging label, a spin label, and any combination thereof.

13. The drug delivery composition of claim 1, comprising a chemically-targeting moiety that is disposed on the surface of the proteolipid nanovesicles, and that comprises a ligand, a dendrimer, an oligomer, an aptamer, a binding protein, an antibody, an antigen-binding fragment thereof, a biomolecule, or any combination thereof.

14. The drug delivery composition of claim 1, wherein the neurosome- or astrosome-specific nanovesicles are about 100 to about 1000 nm in average diameter.

15. The drug delivery composition of claim 1, wherein the synthetic phospholipids are selected from the group consisting of phosphatidylcholine, egg phosphatidic acid, D-Lin-MC3-DMA, 1,2-dioleoyl-sn-glycerophosphocholine (DOPC), 1,2-dioleoyl-sn-glycerophosphoethanolamine (DOPE), 1,2-dipalmitoyl-sn-glycerophosphocholine (DPPC), 1,2-distearoyl-sn-glycerophosphocholine (DSPC), 1,2-distearoyl-sn-glycero-3-phosphoethanolamine-N-[amino(polyethylene glycol)-2000] (ammonium salt) (PEG-2000), 1,2-dipalmitoyl-3-dimethylammonium-propane (16:0 DAP), cholesterol, and any combination thereof.

16. The drug delivery composition of claim 1, wherein the lipid-to-protein ratio is from about 160 to 5 (wt./wt.) to about 300:1.

17. A population of isolated mammalian cells comprising the drug delivery composition of claim 1.

18. A pharmaceutical formulation comprising the drug delivery composition of claim 1, and a pharmaceutically-acceptable buffer, diluent, excipient, or vehicle.

19. A kit comprising the pharmaceutical formulation of claim 18, and instructions for administering the composition to a mammal in need thereof, as part of a regimen for the prevention, diagnosis, treatment, or amelioration of one or more symptoms of a neural disease, a brain dysfunction, an abnormal brain condition, or a brain trauma or injury in the mammal.

20. A method for providing one or more neuroactive agents to a population of neural cells or a brain tissue within the body of an animal, comprising administering to the animal an amount of the pharmaceutical formulation of claim 18, for a time effective to provide the one or more neuroactive agents to the population of neural cells or brain tissue within the body of the animal.

21. A method of administering a diagnostic, therapeutic, or prophylactic agent to one or more neural cells, tissues, organs, or systems of a mammalian subject in need thereof, comprising administering to the subject an effective amount of the pharmaceutical formulation of claim 18.

22. The method of claim 21, wherein the therapeutic agent comprises at least a first siRNA, DNA, ssRNA, RNAi, or any combination thereof.

23. The method of claim 22, wherein the therapeutic agent further comprises at least a first neurotherapeutic agent.
